SEQUENCE LISTING (3 sequences)

SEQ ID NO: 1 - DNA, 597 bp, homo sapiens
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 2 - protein, 198 aa, homo sapiens [sequence omitted]

SEQ ID NO: 3 - DNA, 2794 bp, homo sapiens
agagaaccatcattaattgaagtgagatttttctggcctgagacttgcagggaggcaagaagacactctggacaccactatggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaacttg

(19) United States
(12) Patent Application Publication
(10) Pub. No.: US 2011/0237560 A1
(43) Pub. Date: Sep. 29, 2011

(54) MODULATING AND/OR DETECTING ACTIVATION INDUCED DEAMINASE AND METHODS OF USE THEREOF

(76) Inventors: JAVIER M. DI NOIA, MONTREAL (CA); ALEXANDRE ORTHWEIN, MONTREAL (CA)

(21) Appl. No.: 13/046,214

(22) Filed: Mar. 11, 2011

Related U.S. Application Data
(60) Provisional application No. 61/313,198, filed on Mar. 12, 2010.

(30) Foreign Application Priority Data
Mar. 26, 2010 (CA) 2697887

(51) Int. Cl. A61K 31/568; A61K 31/496; A61K 31/395; A61P 35/00; C12Q 1/68; G01N 33/573

(52) U.S. Cl. 514/171; 514/252.18; 514/183; 435/6.12; 435/7.4

(57) ABSTRACT

A method for stratifying a subject, said method comprising: measuring the AID expression and/or activity in a first sample of the subject, and comparing said expression and/or activity to a reference AID expression and/or activity, wherein an AID expression and/or activity in the first sample of the subject that is higher than the reference AID expression and/or activity is indicative that the subject would benefit from a treatment with at least one Heat Shock Protein 90 (Hsp90) inhibitor.

### CROSS REFERENCE TO RELATED APPLICATIONS

This application claims benefit, under 35 U.S.C. §119(e), of U.S. provisional application Ser. No. 61/313,198, filed on Mar. 12, 2010. All documents above are incorporated herein in their entirety by reference.

### STATEMENT REGARDING FEDERALLY SPONSORED RESEARCH OR DEVELOPMENT

N.A.

### FIELD OF THE INVENTION

The present invention relates to modulating and/or detecting Activation Induced Deaminase (AID) and methods of use thereof. More specifically, the present invention is concerned with methods of stratifying subjects and methods of preventing/treating AID-associated diseases by modulating AID.

### REFERENCE TO SEQUENCE LISTING

Pursuant to 37 C.F.R. 1.821(c), a sequence listing is submitted herewith as an ASCII compliant text file named 765-SEQ LISTING_ST25 created on Mar. 10, 2011 and having a size of 7 kilobytes. The content of the aforementioned file is hereby incorporated by reference in its entirety.

### BACKGROUND OF THE INVENTION

The adaptive humoral immunity of vertebrates allows them to mount a specific response against virtually any foreign substance and organism. To generate the almost infinite number of specific receptors that this requires, B-lymphocytes possess a mechanism for the combinatorial rearrangement of the genes encoding the antibodies. This mechanism, shared with the T-cell receptor, is known as VDJ-recombination and is catalyzed by the endonuclease RAG¹. Unlike the T-cell receptor, the B-cell receptor must increase the affinity for the cognate antigen during the immune response to efficiently eliminate it¹. This is achieved by a second mechanism of genetic modification: somatic hypermutation (SHM), which introduces random mutations over the gene segment that encodes the antibody variable region, thus creating a pool of B-cells displaying related antibodies that compete for the antigen¹⁻³. Darwinian selection of the best ones maturates the overall affinity of the humoral response against the cognate antigen.

The key enzyme behind SHM is Activation Induced Deaminase (AID)⁴, which deaminates deoxycytidine (dC) to deoxyuridine (dU) in the immunoglobulin (Ig) loci. Replication over the dU, or over the intermediates created after specific DNA repair enzymes process the dU, brings about the full spectrum of SHM^{2,5}.

In addition recognition of the dU in the switch regions preceding the exons encoding for each antibody isotype in the heavy chain locus leads to the DNA breaks necessary for class switch recombination (CSR)⁶. Simultaneous targeting by AID of two switch regions at the Ig locus during CSR, allows these non-homologous DNA regions to recombine and loop out the intervening sequence, thereby placing a different constant domain next to the active Ig locus. This process allows the B-cell to switch antibody production from the IgM default isotype to another one (IgG, IgE or IgA), thus acquiring specialized biological properties.

Both SHM and CSR have in common being initiated by an endogenously generated, programmed DNA damage that is resolved by error-prone DNA repair, instead of the usual error-free mechanisms related with uracil in DNA.

### Expression of AID and Associated Diseases

Gene expression regulation is an important step in restricting AID to the relevant tissues and, during normal B-cell development, AID is expressed in germinal center B-cells^{4,7}. Because AID is essential and the only specific factor for antibody diversification (i.e., all proteins known today that act downstream from AID are ubiquitous), any mechanism impinging on the overall steady state levels of AID in B-cells will likely be crucial in balancing an efficient humoral immune response with the associated risk of developing B-cell related pathologies such as B cell lymphomas and leukemias, autoimmune diseases like SLE, atopic allergy. The AID gene can normally also be expressed outside the B-cell compartment (e.g., in the ovaries^{8,9}).

The level of AID expression and/or activity correlates with the efficiency of antibody diversification but also with chromosomal translocations and B-cell lymphomagenesis. This was demonstrated by the proportional defect or increase in these processes observed in AID-haploinsufficient mice^{10,11} or following manipulation of the AID levels by altering its regulation by miRNA¹²⁻¹⁴, or by gene overexpression¹⁵. The existing evidences indicate that AID expression is enough to cause mutations in the Ig genes but also off-target as well as genomic rearrangements including chromosomal translocations. For instance, it was shown in mouse models that AID was required for the c-myc/IgH translocations¹⁶, a hallmark of human Burkitt lymphomas. During this translocation c-myc comes under the influence of the Ig locus enhancers, causing oncogenic expression of the c-myc gene¹⁷. Strikingly, AID's oncogenicity was demonstrated by overexpression of AID in transgenic mice causing tumor formation in different tissues (lung, lymphatic, and liver¹⁸). There is ample evidence that AID can be induced in a variety of human malignant pathologies such as lymphomas^{19,20} and leukemias²¹⁻²³ but also in non-lymphoid solid tumors²⁴⁻²⁶. More specifically, aberrant expression of AID was identified as acting as a mutator enzyme in BCR-ABL1-transformed acute lymphoid leukemia (ALL) cells²².

AID expression could also be normal in B cells and still lead to lymphoma or autoimmune diseases in individuals predisposed by another genetic characteristic (e.g., deficiency in some DNA repair pathways, p53 loss-of-function mutations, etc.). Indeed, there is good evidence that p53 protects B cells from AID-dependent chromosomal translocations and oncogenicity^{15,27}.

### Neoplastic Disease

The transformation of a normal cell into a malignant cell results, among other things, in the uncontrolled proliferation of the progeny cells, which exhibit immature, undifferentiated morphology, exaggerated survival and proangiogenic properties. Once a tumor has formed, cancer cells can leave the original tumor site and migrate to other parts of the body via the bloodstream and/or the lymphatic system by a process called metastasis. In this way, the disease may spread from one organ or part to another non-contiguous organ or part.

The increased number of cancer cases reported around the world is a major concern. Currently there are only a handful of treatments available for specific types of cancer and these treatments provide only limited efficacy and are often associated with toxicity. In addition, one of the biggest concerns of all cancer treatments is the development of chemotherapy resistance.

All steps of cancer progression as well as the development of drug resistance arise as a result of the acquisition of a series of fixed DNA sequence abnormalities, mutations, many of which ultimately confer a growth advantage upon the cells in which they have occurred. Some mutations lead, for example, to the overexpression or constitutive activation of oncogenes not normally expressed by normal mature cells.

### Tumor Profiling

Although the understanding of the molecular pathogenesis of cancer has advanced in the last two decades, risk assessment continues to be solely based on a few clinical parameters. Many studies conducted in recent years support the concept that the prognostic assessment of cancer should routinely include the investigation of molecular biomarkers. Also, because side effects of many treatments are severe, there is a need for targeted therapy. In cancer therapy, the quest for better treatment modalities includes better risk stratification of patients into populations of likely responders to a proposed therapy using small molecules capable of inhibiting hyperactive pathways without adverse effects. In addition, supplementing conventional diagnostics with molecular information should help to identify patients with pre-malignant lesions, patients at risk of developing drug resistance, patients with aggressive tumors for whom maximal therapy is appropriate and others who might survive with less toxic adjuvant therapy of reduced intensity (and thus suffer from less side-effects). Therefore, the development of robust and sensitive assays based on biomarkers linked to appropriate chemotherapeutic agents is certainly a need in cancer.

### Current Needs

There is a need to identify inhibitors of AID in order to modulate AID expression and/or activity in a tissue. There is a particular need to identify inhibitors of AID in order to modulate AID expression and/or activity in a neoplastic or pre neoplastic tissue. There is a need to identify inhibitors of AID in order to control AID expression and/or activity in B cells.

There is a need for identifying AID inhibitors to treat and/or prevent the development of AID-associated diseases in susceptible patients. There is also a need for identifying AID inhibitors to prevent cancer progression and/or development of chemotherapy resistance.

More specifically, there is a need for an improved targeted anti-cancer treatment adapted to specific tumor characteristics. There is thus a need for measuring the level of AID expression and/or activity in a tumor in order 1) to evaluate whether or not a treatment inhibiting AID expression/activity is appropriate and 2) to evaluate the dose of drug necessary to inhibit AID.

There is also a need for identifying AID inhibitors to treat immune system diseases including autoimmune diseases and allergy.

There is also a need for identifying AID inhibitors to treat diseases or hormonal imbalance treated with compounds known to induce AID (e.g., estrogen and proinflammatory cytokines). Without being so limited, estrogen replacement therapy is such a treatment for hormonal imbalance.

### SUMMARY OF THE INVENTION

The present invention shows the link between AID and Heat Shock Protein 90 (Hsp90). The inventors showed that AID is a novel Hsp90 “client” and, as such, physically and functionally interacts with the Hsp90 chaperone pathway. The inventors demonstrated that this interaction is mediated by the N-terminal domain of AID, depends on the ATPase activity of Hsp90 and determines the steady state levels of the bulk of AID. Indeed, inhibition of Hsp90 by a variety of compounds leads to cytoplasmic polyubiquitinylation and proteasomal degradation of AID. This reduction in the level of AID protein is concomitant with a reduction in normal antibody diversification (somatic hypermutation (i.e., Ig SHM), Immunoglobulin gene conversion and class switch recombination), as well as off-target mutation (i.e., any mutation produced by AID at a non Ig gene). The present invention provides compounds that inhibit AID expression and/or activity.

More specifically, in accordance with an aspect of the present invention, there is provided a method for stratifying a subject, said method comprising: measuring the AID expression and/or activity in a first sample of the subject, and comparing said expression and/or activity to a reference AID expression and/or activity, wherein an AID expression and/or activity in the first sample of the subject that is higher than the reference AID expression and/or activity is indicative that the subject would benefit from a treatment with at least one Heat Shock Protein 90 (Hsp90) inhibitor.

In a specific embodiment of the method, when the AID expression in the first sample of the subject is substantially similar to the reference AID expression, the method further comprises the step of: detecting in the first or a second sample of the subject the presence of a loss-of-function mutation, or a deficient expression and/or activity, in at least one gene known to regulate AID mutator activity by controlling or repairing DNA damage, wherein the presence of a mutation in the at least one gene in the first or second sample of the subject is indicative that the subject would benefit from a treatment with at least one Heat Shock Protein 90 (Hsp90) inhibitor.

In accordance with another aspect of the present invention, there is provided a method for the prevention and/or treatment of an AID-associated disease in a subject in need thereof, said method comprising: measuring the level of AID expression and/or activity in a first sample from the subject, comparing said expression and/or activity to a reference AID expression and/or activity, wherein, if the AID expression and/or activity is higher in the first sample from the subject than the reference AID expression and/or activity, an effective amount of an Heat Shock Protein 90 (Hsp90) inhibitor is administered to the patient.

In a specific embodiment of the method, when the AID expression in the first sample of the subject is substantially similar to the reference AID expression, the method further comprises the step of: detecting in the first or a second sample of the subject the presence of a loss-of-function mutation, or a deficient expression and/or activity, in at least one gene known to regulate AID mutator activity by controlling or repairing DNA damage, wherein the presence of a mutation in the at least one gene in the first or second sample of the subject is indicative that the subject would benefit from a treatment with at least one Heat Shock Protein 90 (Hsp90) inhibitor.

In another specific embodiment, the AID-associated disease is cancer and the sample from the subject is pre neoplastic or neoplastic tissue. In another specific embodiment, the cancer is an immune system cancer or a solid tumor. In another specific embodiment, the immune system cancer is chronic myeloid leukemia (CML) or BCR-ABL1-positive acute lymphoid leukemia (ALL). In another specific embodiment, the solid tumor is Helicobacter pylori-associated gastric tumor, liver tumor or colorectal cancer tumor.

In another specific embodiment, the AID-associated disease is an autoimmune disease, and the sample from the subject is a B lymphocyte population of the subject.

In accordance with yet another aspect of the present invention, there is provided a method for preventing drug resistance in a subject having an AID-expressing neoplastic disease, said method comprising: measuring the level of AID expression and/or activity in a tissue sample from the subject, and administering an effective amount of an Hsp90 inhibitor in combination with the drug, to the subject having an AID-positive tissue, whereby the drug resistance is prevented.

In a specific embodiment, the neoplastic disease is chronic myeloid leukemia. In another specific embodiment, the drug is imatinib.

In another specific embodiment of the method, the Hsp90 inhibitor is a geldanamycin analog. In another specific embodiment, the geldanamycin analog is 17-(Allylamino)-17-demethoxygeldanamycin (17-MG), 17-(Dimethylaminoethylamino)-17-demethoxygeldanamycin (17-DMAG), nab-17-AAGs, NXD30001 or CNF1010.

In another specific embodiment, the administration is a monotherapy. In another specific embodiment, the method further comprises administration of at least one other therapy to the subject. In another specific embodiment, the at least one other therapy comprises at least one further AID inhibitor. In another specific embodiment, the at least one AID inhibitor is not an Hsp90 inhibitor. In another specific embodiment, the method further comprises administration of at least one further anticancer treatment. In another specific embodiment, the subject is undergoing a therapy that comprises the administration of least one compound that increases AID expression and/or activity in a normal tissue. In another specific embodiment, the compound is estrogen.

In accordance with yet another aspect of the present invention, there is provided a method for adjusting a dose of a Hsp90 inhibitor in a treatment, said method comprising: measuring the level of AID expression and/or activity in a sample of a subject treated with an Hsp90 inhibitor, comparing said expression and/or activity to a reference AID expression and/or activity from the subject at an earlier time, and administering to the subject having a substantially similar or higher AID expression and/or activity than the reference AID expression and/or activity an increased dose of the Hsp90 inhibitor.

In accordance with a further aspect of the present invention, there is provided a kit for preventing and/or treating an AID-associated disease or for stratifying a subject having an AID-associated disease comprising an AID ligand and a Heat Shock Protein 90 (Hsp90) inhibitor.

In another specific embodiment, of the kit, the AID-associated disease is a neoplastic disease and further comprising a further antitumoral agent.

### BRIEF DESCRIPTION OF THE DRAWINGS

In the appended drawings:

FIG. 1 shows the association between AID and Hsp90. (A) Several members of the Hsp90 pathway copurify with AID. AID-Flag/HA from stably expressing Ramos B-cell line was pulled down by two consecutive immunoprecipitations using anti-Flag and anti-HA and eluted with the specific peptides. The purified material was fractionated by 4-20% SDS-PAGE. The gel was cut into 20 slices, submitted to tryptic digestion, the peptides analyzed by mass spectrometry and compared to a database. The proteins relevant for this work are indicated next to the bands from where they were identified. One of two experiments is shown. (B) AID interacts with endogenous Hsp90 in Ramos B-cells. GFP and AID-GFP were immunoprecipitated from extracts of stably expressing Ramos B-cells. Following SDS-PAGE, eluates were analyzed by western blot with anti-GFP and anti-Hsp90 antibodies. Aliquots (5%) of the total-cell extracts were probed with anti-Hsp90 as loading and expression control. One of three identical experiments is shown. (C) AID interacts similarly with the two major isoforms of Hsp90. The physical association between Hsp90-alpha or -beta and AID were monitored by transiently cotransfecting HEK293T cells with AID-GFP and Flag-Hsp90alpha or myc-Hsp90beta, immunoprecipitating with anti-GFP and analyzing the eluates by western blot with anti-myc and anti-Flag The filters were then probed with anti-Hsp90 (recognizes both isoforms) to verify that the overall Hsp90 level was similar in both cells after transfection and with anti-GFP to confirm similar immunoprecipitation of the bait. A2-GFP was used as a negative control cotransfected with both tagged Hsp90 isoforms. One of two identical experiments is shown;

FIG. 2 shows the specific and localized binding of AID to Hsp90. (A) Hsp90 interacts specifically with AID within the AID/APOBEC family. Lysates from HEK293T cells cotransfected with myc-tagged Hsp90beta and Flag-tagged versions of the indicated AID/APOBECs (i.e. APOBEC1 (identified as A1 in FIG. 2A), APOBEC2 (identified as A2 in FIG. 2A) and APOBEC3G (identified as A3G in FIG. 2A)) were immunoprecipitated using anti-Flag antibodies and analyzed by western blot with anti-myc to verify the presence of Hsp90beta and anti-Flag to ascertain the immunoprecipitation of all the baits. AID migrates slightly higher than APOBEC1 due an additional HA tag²⁸. One of four experiments yielding identical results is shown. (B) Schemes of the AID-APOBEC2 chimerical proteins used to yield results presented in FIG. 2 C and D. The black lines identify the fragment of AID replaced by the homologous positions from APOBEC2 as determined by sequence alignment and structural prediction²⁹. For instance in chimera #1, the fragment 19-57 of AID was replaced by amino acids 60-96 from APOBEC2; while in chimera a only amino acids 34-36 from AID were replaced by the corresponding APOBEC2 positions. These proteins have been described 28,29. Secondary structure for APOBEC2 (designated A2) (experimentally determined in ref³⁰) and AID (predicted by molecular modeling in ref²⁸) is indicated below each protein scheme. Rectangles indicate alpha helixes (a1, a2, etc) and arrows beta strands (b1, b2, etc). (C) The N-terminal domain of AID mediates the binding to Hsp90beta. Lysates from HEK293T cells cotransfected with myc-tagged Hsp90beta and Flag-tagged versions of the indicated AID-APOBEC2 chimeras were immunoprecipitated with anti-Flag and analyzed by western blot using anti-myc antibodies. Filters were then probed with anti-Flag to confirm similar immunoprecipitation of the bait. One representative out of three experiments performed is shown. (D) Smaller substitution of AID residues only partially abrogate the interaction with Hsp90beta. Experiments were performed as in (C). One of two experiments is shown. (E) The position of the tag on AID does not affect the association to Hsp90. HEK293T cells were cotransfected with myc-tagged Hsp90 and GFP-AID, AID-GFP or A2-GFP, GFP as controls. Anti-GFP immunoprecipitates were analyzed by western blot with anti-myc and anti-GFP. One out of three identical experiments is shown. (F) AID oligomerization or phosphorylation are not required for Hsp90 interaction. Interaction of Hsp90 with AID mutants carrying the F46A/Y48A/R50G/N51A simultaneous mutations (FYRN), previously shown to be defective for oligomerization (21) or T27A and T38A phospho-null mutations (T27/38), was tested as in (E). In all panels aliquots (5%) of the total cell extracts were probed with anti-myc to control for expression levels of Hsp90;

FIG. 3 shows that Hsp90 maintains the steady-state levels of AID. (A) The ATPase activity of Hsp90 is essential for its interaction with hAID. Ramos cells stably expressing AID-GFP or GFP alone were treated with 2 microM geldanamycin (GA) or DMSO for 2 h before harvesting, lysis and anti-GFP immunoprecipitation. Eluates were fractionated on SDS-PAGE and blots were probed with anti-Hsp90 and anti-GFP. Aliquots (5%) of the extracts were probed to control for expression levels of Hsp90. One of two experiments is shown. (B) Hsp90 inhibition results in decreased steady-state levels of endogenous AID. Human, mouse and chicken B-cell lines (Ramos, CH12-F3 and DT40, respectively) were treated with 2 microM GA or DMSO and harvested at the indicated time points. The expression level of the indicated proteins was analyzed by Western Blot in total or nuclear (where indicated) extracts. CH12-F3 cells were pretreated for 24 h with IL-4/TGFb-1/anti-CD40 to induce AID and stimulate transcription from an intronic promoter at the Ig locus that is necessary for CSR. The human and chicken cell lines used have constitutive expression and did not need induction. One out of three experiments is shown for each cell line. (C) Hsp90 inhibition leads to lower AID steady state levels in primary human B cells. Resting B cells were purified from blood of three donors, treated as indicated 4 days post-activation with IL4/anti-CD40 and analyzed as in (B). The GA derivative 17-(Allylamino)-17-demethoxygeldanamycin (17-MG) was used in this case because of concerns on the viability of primary B cells when incubated with GA that is more toxic in cell culture (see below). (D) AID stabilization by Hsp90 requires protein-protein interaction. Ramos cells stably expressing GFP, AID-GFP or chimeras AID-A2 #1 or #2 (as described in FIG. 2 B) were treated in triplicate with 2 microM GA or DMSO. The GFP mean fluorescence intensity (MFI), a measure of GFP signal by flow cytometry, was monitored at various time points by flow cytometry. MFI values normalized to t₀=100% are plotted over time. Dead cells were excluded by propidium iodide staining. (E) Hsp90 plays a role in stabilizing fully synthesized AID. AID-GFP was monitored as in (D) except that Ramos cells were pretreated with 100 ng/mL Cycloheximide (CHX) for 30 min to inhibit protein synthesis before Hsp90 inhibition. Pretreatment with CHX allowed to follow the pool of AID that had already been synthesized and not the nascent AID that might be more sensitive to folding requirement. One of five independent experiments is shown. (F) Hsp90 inhibition destabilizes AID-GFP in primary mouse B-cells. Purified naïve B-cells from aid−/− mice were activated and retrovirally transduced with mouse AID-GFP. Two days post-transduction, cells were treated with 2 microM GA or DMSO and the GFP MFI monitored as in (C). (D) and (F) are representative of three different experiments. Two asterisks indicate statistical significance evaluated by Student t-test with P<0.01;

FIG. 4 shows that Hsp90 inhibition results in cytoplasmic ubiquitinylation and degradation of AID by the proteasome. (A) AID degradation following Hsp90 inhibition is distinct from nuclear AID degradation. Ramos cells stably expressing AID-GFP were treated in triplicate with DMSO (Ctrl), 2 microM GA and/or 50 ng/mL leptomycin B (LMB) and the GFP signal was monitored over time by flow cytometry. The MFI normalized to t₀=100% is plotted for each treatment. Dead cells were excluded by propidium iodide staining. One out of five identical experiments is shown. (B) Newly synthesized AID does not show the additive effect of Hsp90 inhibition and nuclear export inhibition. Ramos cells stably expressing AID-GFP pretreated with 100 ng/mL cycloheximide (CHX), a known protein synthesis inhibitor, for 1 h were treated and analyzed as in (A). One out of four identical experiments is shown. (C) Cytoplasmic destabilization of AID following Hsp90 inhibition. Analogous experiments to those in A and B were performed on Ramos cells stably expressing GFP-AID, which was previously shown to be unable to enter the nucleus (probably because the N-terminal fusion of GFP to AID masks the NLS)²⁸. One out of three identical experiments is shown. (D) AID degradation following Hsp90 inhibition requires the proteasome. Ramos cells stably expressing AID-GFP were treated with DMSO (Ctrl), 2 microM 17-MG and 10 microM MG132, a known specific proteasome inhibitor, as indicated. The GFP signal was monitored and plotted as above. One out of five experiments is shown. (E) Lower AID steady-state levels induced by Hsp90 inhibition can be blocked by proteasome inhibition in B cell lymphoma lines. Human and chicken B-cell lines (Ramos and DT40 respectively) were treated with 2 microM GA and 10 microM MG132 and subsequently harvested as indicated. The cells were lyzed, fractionated on SDS-PAGE and blotted. Blots were probed with anti-AID and anti-actin. One out of two experiments is shown for each cell line. (F) Hsp90 inhibition enhances AID polyubiquitination. Ramos B-cells stably expressing AID-GFP were treated with 10 microM MG132 and 2 microM GA for 5 h as indicated, lyzed and subsequently immunoprecipitated. Immunoprecipitates were analyzed by western blot using anti-GFP and anti-ubiquitin antibodies. In the middle panel, the same experiment was performed with primary mouse B cells transduced with mouse AID-GFP. In the lower panel, the relative amount of polyubiquitinated AID was quantified by densitometry using ImageQuant™ and the relative value of three independent experiments for each Ramos, HeLa and primary mouse B cells was plotted+SD. Two asterisks indicate statistical significance evaluated by Student t-test with P<0.01;

FIG. 5 shows that Hsp90-associated E3 ubiquitin ligase CHIP can reduce the levels of AID. (A) AID interacts with CHIP. HeLa cells stably expressing AID-GFP were transfected with myc-CHIP and treated for 5 h with DMSO, 2 microM GA, 50 ng/mL leptomycin B (LMB) and 10 microM MG132 in the combinations indicated. Cells were harvested, lysed and immunoprecipitated with anti-GFP. Eluates were fractionated on SDS-PAGE and filters were probed with anti-GFP and anti-myc. Aliquots (5%) of the extracts were probed to control for expression levels of myc-CHIP. One of two identical experiments is shown. (B) Overexpression of CHIP in B-cells results in decreased steady-state levels of endogenous AID. Ramos cells lines expressing myc-CHIP or pcDNA3.1 control were established by transfection and G418 selection. Subclones from control population and three independent myc-CHIP transfectants were obtained by limiting dilution. AID levels were estimated by western blot using anti-AID for each subclone after cell culture expansion. Anti-actin was used as loading control and anti-myc to confirm the expression of CHIP. Three representative subclones from each original transfectant are shown. (C) AID levels for all subclones obtained as in (B) were estimated from non-saturated western blots using ImageQuant™ software. The signal was normalized to each corresponding actin signal obtained from equivalent exposures and plotted. Median values are indicated. Significance was evaluated by Student t-test, P<0.01. Subclones derived from each independent myc-CHIP transfectant are distinguished by different symbols (Myc-CHIP CL11 shown as circles; Myc-CHIP CL24 shown as squares; and Myc-CHIP CL25 shown as triangles).

FIG. 6 shows reduced antibody diversification in chicken and mouse B-cells chronically treated with Hsp90 inhibitors. (A) Diminished Ig gene conversion in DT40 cells treated with GA. The proportion of sIgM-gain cells arising from sIgM⁻ DT40 cell populations after 3 weeks of expansion in the presence of DMSO or two different concentrations of GA is plotted (left panel). The median obtained for populations treated in each condition is indicated. The level of AID was estimated by western blot for each population at the end of the experiment. The relative level of AID was calculated by normalizing to actin levels after quantitation of non-saturated western blot signals. Mean+SD values for the seven populations in each condition are plotted (middle panel). The western blots for each group are shown (right panel). (B) Diminished Ig gene conversion in DT40 cells treated with 17-AAG. An identical experiment to (A) was performed except that the less toxic 17-AAG was used to inhibit Hsp90. The fluctuation analysis for IgM-gain (left panel) and quantitation of AID levels (middle panel) are shown. The effect of the two 17-AAG concentrations used on DT40 growth was monitored by calculating the total number of cells in populations originating from 10⁵ cells (left panel). The data plotted is the mean+SD of triplicate cultures for each condition. (C) Diminished somatic hypermutation in DT40 cells treated with 17-AAG. The proportion of sIgM-loss cells arising from sIgM⁺ phiV⁻ AID^{R} DT40 cell populations after 3 weeks of expansion in the presence of DMSO or two different concentrations of 17-AAG is plotted for 6 populations grown in each condition (left panel). The quantitation of AID levels (middle panel) and cell growth curves (right panel) were done as in (B). (D) Chronic Hsp90 inhibition results in a reduced class-switch recombination. CH12F3-2 mouse B cells were activated with IL-4, TGFbeta1 and agonist anti-CD40 to induce switching to IgA and cultured in the presence of DMSO or the indicated concentrations of 17-AAG. The cells were stained with CFSE prior activation to be able to monitor the number of divisions. Representative plots of the proportion of IgA+ cells in each population after 3 days (left) and CFSE profiles (middle) are shown. For each cell division the proportion of sIgA⁺ cells was calculated and the results from four experiments are summarized in the plot as the mean+SD values (right). One or two asterisks indicate statistical significance evaluated by Student t-test with P<0.05 or P<0.01, respectively;

FIG. 7 shows that acute inhibition of Hsp90 impairs antibody diversification in primary mouse B cells. (A) The AID levels in CH12F3-2 mouse B-cells as determined by western blot at different times (0-4 days) post-activation with IL-4, TGFbeta-1 and agonist anti-CD40. (B) Acute Hsp90 inhibition results in reduced class-switch recombination in CH12F3-2 mouse B cells. The cells were stained with CFSE and activated for switching to IgA as above and were treated by 12 h with 2 microM 17-AAG either at day 1 (D+1) or 2 (D+2) post-activation and then returned to normal medium. The proportion of sIgA⁺ cells per cell division determined by flow cytometry is plotted for each cell division below the corresponding CFSE signal range for a representative experiment (left). The results from four experiments are summarized by plotting the mean proportion of sIgA⁺ cells per cell division+/−SD. (C) Acute Hsp90 inhibition reduces class-switch recombination in primary mouse B cells. Purified naïve splenic mouse B-cells were stained with CFSE and stimulated with IL-4 and LPS to induce switching to IgG1. The cells were treated with 17-MG as in (B) and the proportion of sIgG1⁺ cells per division determined. Flow cytometry profiles for one representative mouse is shown (left). Data from five mice is plotted as the relative mean proportion+/−SD of sIgG1⁺ cells for each cell division. To be able to compare all the mice accounting for the inter assay variability, all data points were normalized to the % of IgG1+ cells in the control at cell division 3 defined as 1;

FIG. 8 shows that the treatment of cells with a Hsp90 inhibitor reduces AID off-target mutations. (A) BCR-ABL1+CML K562 cells were transduced with retroviral vector control expressing GFP or with retroviral vector encoding AID linked to GFP expression by an internal ribosomal entry site (ires). Mixed populations of transduced and non-transduced cells were cultured in the presence of DMSO (Diamonds), 2 microM Imatinib (triangles), 0.1 microM 17-MG (squares) or 2 microM imatinib plus 0.1 microM 17-MG (circles). The proportion of GFP+ cells was followed over time by flow cytometry. An identical experiment with K562 expressing only GFP was done as control (inset). Data were plotted as the mean GFP+/GFP− ratio from triplicate populations+/−SD, relative to the initial ratio set as 1. Two independent experiments are shown differing only in AID protein level expression from having or not a consensus Kozak sequence before the AID start codon. (B) Presence of a Kozak sequence leads to higher AID expression in transduced CML K562 cells. Parental K562 cells (−), and sorted GFP+ populations transduced with retroviral vector encoding AID-ires-GFP differing only in the presence of the Kozak sequence were analyzed by Western Blot with an anti-AID antibody and anti-Actin as a loading control. (C) Hsp90 inhibition results in decreased levels of AID in transduced CML cells. Sorted GFP+ cells transduced with retroviral vector encoding AID-ires-GFP as in (A) were cultured with 0.1 microM 17-MG for the indicated times. The levels of the indicated proteins were analyzed by Western blot in total extracts using anti-AID and anti-ABL1. One of two experiments is shown. (D) Hsp90 inhibition prevents AID-mediated increase in imatinib IC₅₀ (which is the half maximal inhibitory concentration necessary to cause cell death). GFP+K562 populations were sorted after expansion from (A). Cell viability after culture for 2 days with different concentrations of imatinib was determined using an MTS (3-(4,5-dimethylthiazol-2-yl)-5-(3-carboxymethoxyphenyl)-2-(4-sulfophenyl)-2H-tetrazolium) reduction colorimetric assay. The relative mean absorbance at 490 nm+/−SEM of duplicate wells (untreated cells set as 100%) is plotted for each concentration for cell populations transduced with the retroviral vector encoding KozakAID-ires-GFP that had been treated in (A) with DMSO (Diamonds), 2 microM imatinib (triangles), 2 microM imatinib plus 0.1 microM 17-MG (circles), or 17-MG (squares). The parental K562 cells were included in this assay. One of two independent experiments is shown. (E) BCR-ABL1 (exon13 of BCR and exon 9 of ABL10) was PCR amplified from cells expressing GFP control or AID-ires-GFP and expanded under the indicated conditions. Mutations, determined relative to the consensus of all sequences, are indicated on schemes of the 700 bp ABL1 region that was directly sequenced from the PCR product. Thin vertical bars represent mutations at A:T bp, whereas thick bars represent mutations at C:G pairs. Mutations previously described to confer imatinib resistance¹⁰¹ are identified by an asterisk and indicated below the sequence stack;

FIG. 9 shows that the treatment of cells with a Hsp90 inhibitor reduces the number of AID-mediated mutations in BCR-ABL1. The BCR-ABL1 (exon 13 of BCR and exon 9 of ABL1) was RT-PCR amplified from single cell clones, and a fragment of 700 bp in the ABL1 kinase domain was directly sequenced. The number to the left of the mutation indicates the mutated position with respect to the ABL1 open reading frame. The mutated base is underlined within the affected codon. Mutations at C:G pairs and amino acid substitutions previously described to confer clinical imatinib resistance¹⁰¹ are highlighted in bold;

FIG. 10 shows the expression levels of AID, Hsp90 and CHIP in various B cells. (A) The parental Ramos B-cells and its derived lines stably expressing AID-Flag/HA (AID-F/H) and AID-GFP were lysed and fractionated on SDS-PAGE. Blots were probed with anti-AID to compare the level of expression each transgenic AID compared to the endogenous enzyme. AID levels were quantified using Imagequant™ and the ratio (R) of tagged to endogenous AID is indicated. (B) Hsp90 and CHIP levels were estimated in human Ramos and chicken DT40 B cell lines. Lysates from both cell lines were analyzed by western blot using anti-Hsp90alpha, anti-Hsp90beta, anti-CHIP and anti-actin as a loading control. Since anti-Hsp90alpha and anti-CHIP are monoclonal antibodies raised against human proteins, apparent differences in expression between Ramos and DT40 cells might just reflect variations in the chicken epitopes. (C) Hsp90 and CHIP levels were estimated in purified naïve mouse B cells activated with IL-4/LPS. As above, purified mouse B cells were harvested at each time point indicated, lysed and subsequently analyzed by western blot using anti-Hsp90alpha, anti-Hsp90beta, anti-CHIP and anti-actin as a loading control;

FIG. 11 shows that AID dependence on Hsp90 is unaffected by Protein Kinase A (PKA) inhibition or activation. (A) Ramos cells stably expressing AID-GFP were treated with the PKA inhibitor H-89 (10 microM) before treating the cells with DMSO or 2 microM GA. AID-GFP was followed by flow cytometry and the MFI (normalized to the t=0 signal) plotted at different times for each treatment. (B) Identical experiments to (A) using the adenylate cyclase activator Forskolin (50 microM) in combination with the phosphodiesterase inhibitor 3-Isobutyl-1-methylxanthine (IBMX; 100 microM) to boost cAMP levels. This treatment increased the level of GFP and AID-GFP in Ramos cells. The reasons behind this increase are unknown but while the GFP increase is unaffected by Hsp90 inhibition, a similar increase in AID-GFP is totally prevented by GA, confirming the dependence of AID on Hsp90. In both cases dead cells were excluded with propidium iodide staining and the data shown are mean+/−SD of triplicate experiments;

FIG. 12 shows that inhibition of Hsp90 has little effect on AID compartmentalization. HeLa cells were transfected with untagged hAID and the cells were treated 48 h later with Hsp90 and/or nuclear export and/or proteasome inhibitors (LMB and MG132, respectively) (Ctrl=DMSO, 2 microM GA, 50 ng/mL LMB, 10 microM MG132, alone or in the indicated combinations). AID localization was monitored by IF. The difference between (2 h GA+2 h LMB) and (2 h GA+LMB) is the timing of addition of LMB; in the first case, cells were pretreated with GA before addition of LMB whereas in the latter case both drugs were added simultaneously;

FIG. 13 shows that the effect of Hsp90 inhibitor on AID stability is dose-dependent and conserved in chicken and non-B cells. (A) DT40 cells and (B) Hela aid−/− cells stably expressing AID-GFP were treated with the indicated combinations of DMSO (Ctrl), 2 microM GA, 50 ng/mL leptomycin B (LMB), a nuclear export inhibitor and/or 10 microM MG132, a proteasome inhibitor. The GFP signal was monitored by flow cytometry and the MFI normalized to the signal at t0 for each treatment. Dead cells were excluded with propidium iodide staining. Three identical experiments for each were averaged and the resulting mean+/−SD are plotted for each time. (C) Ramos cells stably expressing AID-GFP were treated with DMSO (Ctrl) or the indicated concentrations of GA and the GFP signal monitored over time by flow cytometry. The MFI at each time point normalized to the t=0 signal. Dead cells were excluded with propidium iodide staining. Three identical experiments were averaged and the resulting mean+/−SD are plotted for each time; and

FIG. 14 shows A) the nucleotide sequence (cDNA) (SEQ ID NO: 1, genebank accession NM_{—}020661); B) the amino acid sequence (SEQ ID NO: 2, UniProtKB/Swiss-Prot Q9GZX7-1) of human AID; and C) the Genebank™ information about human AID providing the amino acid sequence (SEQ ID NO: 2) and full gene sequence (SEQ ID NO: 3).

### DESCRIPTION OF ILLUSTRATIVE EMBODIMENTS

Drugs inhibiting AID are described as well as methods for the stratification of subjects and methods for the prevention and treatment of AID-associated diseases based on measuring and inhibiting AID in subject's samples such as fluids, tissues and tumors.

### Neoplastic Diseases and AID

By the terminology “neoplastic disease” or “invasive disease” is meant herein to refer to a disease associated with new growth of any body tissue. A neoplastic tissue according to the invention is derived from a pre neoplastic tissue and may retain some characteristics of the tissue from which it arises but has biochemical characteristics that are distinct from those of the parent tissue. The tissue formed due to neoplastic growth is a mutant version of the original tissue and appears to serve no physiologic function in the same sense as did the original tissue. It may be benign or malignant (e.g., cancer).

Cancer is defined herein as a disease characterized by the presence of cancer cells which possess two heritable properties: they and their progeny are able (1) to reproduce unrestrained in defiance of the normal restraints (i.e., they are neoplastic) and (2) invade and colonize territories normally reserved for other cells (i.e., they are malignant). Invasiveness of cancer cells usually implies an ability to break loose, enter the bloodstream or lymphatic vessels, and form secondary tumors, or metastases at the other distant sites in the body. The term “cancer cells” refers herein to a cluster of cancer or tumor cells showing over proliferation by non-coordination of the growth and proliferation of cells due to the loss of the differentiation ability of cells. The terms “cancer cell” and “tumor cell” are used interchangeably herein.

AID is not systematically expressed in all cancers nor in all tumors of a defined cancer type. For example, AID expression variations were observed amongst gastric adenocarcinomas³¹ and cholangiocarcinomas³². In another example, CML cells in lymphoid blast crisis (fatal within weeks and months) as opposed to chronic phase (indolent chronic phase standing for years), express AID at high levels²³. Also, only a fraction of B-chronic lymphocytic leukemia (B-CLL) cells express AID, which is associated with poor prognosis (although it is not on its own an independent predictor of poor prognosis)³³.

Several data strongly suggest the involvement of AID in inflammation-associated carcinogenesis in humans (Reviewed in ³⁴). For instance, aberrant AID expression was revealed in colonic mucosa and cancer tissues of patient with inflammatory bowel disease, but not in normal colonic mucosa.

AID expression in tumors correlates with the presence of somatic mutations in oncogenes, which show the hallmarks of AID-mediated mutation. This has been demonstrated in CML²² and in gastric cancer samples from Helicobacter pylori infected patients²⁶.

Therefore, an aberrant AID expression and/or activity in a human tissue can be indicative that said tissue may become neoplastic and/or progress to a malignant/cancerous state. It may thus be desirable to inhibit aberrant AID expression and/or activity in subjects having or susceptible to develop a neoplastic disease.

### Genes Regulating AID Mutator Activity in B Cells by Controlling or Repairing DNA Damage

The AID mutator activity is modulated by several genes known to control/prevent/repair AID-mediated mutations and/or AID-mediated antibody diversification. Amongst them, protein 53 (p53), ataxia telangiectasia mutated (ATM), Nijmegen breakage syndrome 1 (Nbs1) and Alternate-reading-frame tumor suppressor (p19(Arf))²⁷, p53 upregulated modulator of apoptosis (PUMA), bcl-2 interacting mediator of cell death (Bim) and protein kinase C, delta (PKCdelta)³⁶ are involved in the control of DNA damage, genomic instability checkpoints and induction of apoptosis. Other genes whose deficiency has been shown to have a synergistic effect with the presence of AID on increasing off-target mutations include the DNA repair enzymes that can recognize uracil in DNA. Examples of DNA repair enzymes include uracil DNA-glycosylase (UNG2)³⁶⁻³⁸, which starts base excision repair; MSH2 and MSH6^{36,37,39}, a mismatch recognition heterodimer that initiates mismatch repair, as well as downstream components of those pathways, such as the DNA polymerase Beta⁴⁹.

Therefore, the deficient expression and/or activity in a B cell population of a gene regulating AID mutator activity by controlling or repairing DNA damage (e.g., a decrease in the p53 DNA damage controlling activity) may be indicative of a predisposition to B cell pathologies due to an increased activity of AID.

B cell leukemias and lymphomas expressing a level of AID expression similar to that observed in normal B cells but combined to deficient expression and/or activity of a gene regulating the AID mutator activity by controlling or repairing DNA damage (e.g., a decrease in p53 DNA damage controlling activity) is also indicative that said cancer may progress to a more malignant state or is susceptible to develop resistance to drug treatment due to an increase activity of AID. It may thus be desirable to inhibit AID in those subjects having B cells in which expression and/or activity of genes regulating AID mutator activity is decreased.

The level of expression of genes (RNA and/or protein) regulating the AID mutator activity can be measured using a variety of assays such as those described below for AID.

Alternatively, the detection of a genomic loss-of-function mutation could be used to measure a decrease in the expression and/or activity of the genes regulating the AID mutator activity by controlling or repairing DNA damage (e.g., loss-of-function mutation at the p53 locus). Genetic loss-of-function mutations are DNA modifications (e.g., deletions, missense substitutions) leading to a decrease in expression and/or activity of a specific gene. For instance, the TP53 (tumor protein 53) gene is the most frequently mutated gene in sporadic cancers. Germline mutations have also been reported in over 500 cancer-prone families. Both somatic and germline mutations are compiled in a worldwide database at the International Agency for Research on Cancer¹⁰⁰. Most p53 loss-of-function mutations result in missense substitutions that are scattered throughout the gene but are particularly dense in exons 5-8, encoding the DNA binding domain.

Several well-known examples of loss-of-function mutations in genes regulating the AID mutator activity by controlling or repairing DNA damage were reported. As reviewed by Coll-Mulet et al.⁴², chronic lymphocytic leukaemia (CLL) is a genetically heterogeneous disease. As detected by the interphase cytogenetic fluorescence in situ hybridization (FISH) approach, the most frequent genetic alterations in the prognosis of B-cell chronic lymphocytic leukemia (B-CLL) patients involve deletions in 17p13 (TP53) and 11q22-q23 (ATM). The importance of studying p53 pathway defects in chronic lymphocytic leukemia (CLL) has been promoted by the demonstration of the fundamentally different clinical course of patients with 17p deletion. The observation of resistance to chemotherapy and mutation of the remaining TP53 allele explains the clinical presentation of CLL with 17p deletion⁴³. In addition, UNG-deficient mice are predisposed to B-cell lymphomas, likely as a consequence of AID expression⁴¹.

The most relevant techniques used for detection of genetic alterations in B cells include, amongst others, comparative genomic hybridization (CGH) and FISH, as well as PCR-based techniques coupled with DNA sequencing or multiplex ligation-dependent probe amplification (MLPA) analyses 42.

### AID and Cancer Progression

Several papers show that in several cancer types (e.g., CML, ALL and B-CLL), AID expression and poor prognosis correlate. One paper also showed AID expression during progression in follicular lymphoma FL suggesting that AID+ clones may outgrow the population and that those cases have more advanced states of the disease^{22,23,33,44}.

### AID and Drug Resistance in Cancer

Tumor resistance (low or no sensitivity to treatment) is a major obstacle to chemotherapy. To date, a variety of mechanisms are known to explain how tumors acquire such resistance. At least for chronic myeloid leukemia (CML) the expression of AID has important consequences by driving the mutations leading to resistance to a therapeutic drug. Indeed, Klemm et al.²³ have published evidences linking AID activity and resistance to Imatinib in CML treatment.

### Autoimmunity and AID

Autoimmunity encompasses a broadly defined area of clinical pathologies that stem from abnormalities in numerous systemic, cellular, and molecular mechanisms, a subset of which are B cell-related autoimmune^{45,48}. In systemic lupus erythematosus, abnormalities in B cell development and the production of autoreactive antibodies play an important pathological role. Overexpression of AID in autoimmune-prone mice induced a more severe systemic lupus erythematosus-like phenotype⁴⁷, whereas breeding AID-deficient mice with autoimmune-prone MRL/lpr mice significantly reduced the onset and extent of disease⁴⁸, indicating that alterations in AID can change the severity of B cell autoimmunity. There are several hypotheses on how unregulated AID can affect autoimmunity in addition to overstimulation of SHM and CSR, e.g., debilitating mutations in the signaling pathways, inactivation of tumor suppressors or proapoptotic genes, or alterations that activate oncogenes or antiapoptotic genes (for review see ⁴⁹).

As used herein, “Autoimmune disease” refers to illnesses that occur when the body tissues are attacked by its own immune system. The immune system is a complex organization within the body that is designed normally to “seek and destroy” invaders of the body, including cancer cells. Patients with autoimmune diseases frequently have unusual antibodies circulating in their blood that target their own body tissues. Examples of autoimmune diseases include Systemic Lupus Erythematosus (SLE), Sjogren syndrome, Hashimoto thyroiditis, Rheumatoid Arthritis (RA), juvenile (type 1) diabetes, polymyositis, scleroderma, Addison disease, vitiligo, pernicious anemia, glomerulonephritis, Multiple Sclerosis (MS), Crohn's disease and pulmonary fibrosis. Autoimmune diseases are more frequent in women than in men. It is believed that the estrogen of females may influence the immune system to predispose some women to autoimmune diseases. Autoimmune diseases that occur more frequently in women than men include RA and SLE. The Relapsing-Remitting and Secondary Progressive forms of MS are nearly twice as common in women as in men although the Primary Progressive form is equally common in men as women.

An aberrant AID expression and/or activity in human B cells may thus be indicative of a predisposition to develop an autoimmune disease.

AID is necessary for CSR to IgE, the immunoglobulin that mediates allergy. It would therefore be useful to administer Hsp90 inhibitors to inhibit AID and in turn reduce production of IgE, thus reducing the severity of atopic allergic reactions⁴⁹⁻⁵¹.

Normal AID expression in B cells combined to a deficient expression and/or activity of genes regulating AID mutator activity by controlling or repairing DNA damage may also be indicative of predisposition to autoimmune disease.

### Estrogen and Increased AID Expression

Pauklin et al.⁹ demonstrate that the estrogen-estrogen receptor complex binds to the AID promoter, enhancing AID messenger RNA expression, leading to a direct increase in AID protein production and alterations in SHM and CSR at the Ig locus. The authors propose that the reported effect of sex-hormones on autoimmunity could partially be through AID transcription resulting in a modified or exacerbated antibody response⁹ as it has been shown in mice⁴⁷. More importantly, this paper directly shows that the increase in AID mRNA production by estrogen is readily detectable outside the immune system, namely in breast and ovarian tissue (>20-fold increase). Enhanced translocations of the c-myc oncogene showed that the genotoxicity of estrogen via AID production was not limited to the Ig locus. The findings suggest a link between estrogen and DNA damage that could be important in the etiology of cancers affecting estrogen-responsive tissues through induction of AID and subsequent increase in genome instability. Such link suggests that it might be advantageous to screen for AID expression in women with preneoplastic manifestations in estrogen-responsive tissues or subjected to hormonal treatments including estrogen. Therapy could potentially be combined with treatments that decrease AID expression and/or activity for reducing such pathological side effects of estrogen.

### Infectious Agents, Cytokines and AID

Some infectious agents normally associated with cancer were reported to lead to AID activation and could thus play a role in AID associated diseases. This is the case for hepatocellular carcinoma-associated HCV^{52,53}; gastric cancer-associated H. pylori²⁶, AIDS-associated non-Hodgkin's B cell lymphoma (NHL) in which, after HIV infection, an elevated level of AID in peripheral blood precedes the onset of NHL⁵⁴, and sporadic NHL associated with EBV⁵⁶. Although these are all association studies, they correlate with the presence of aberrant SHM in oncogenes, caused by AID. Furthermore, transgenic AID was shown to be implicated in the pathogenesis of hepatitis C virus (HCV)-induced human hepatocellular carcinoma (HCC)⁵⁶.

Induction of AID expression was found to depend on the NF-kappaB activation by Helicobacter pylori and HCV core protein. Recent studies have also revealed that AID is aberrantly expressed in non-lymphoid cells not only as a result of infections but also following stimulation with various proinflammatory cytokines (e.g., TNFalpha, IL-1 beta), leading to the generation of off-target gene mutations (Reviewed in ³⁴). Many cancers, some of which are caused by infectious agents, are linked to chronic inflammation.

The present invention provides the novel and unexpected observation that AID expression and activity are sensitive to Hsp90 inhibitors. Indeed, the present invention demonstrates a dose dependant reduction of AID activity (e.g., somatic hypermutation and class switch recombination activities) upon treatment of cells with Hsp90 inhibitors (i.e. 17-MG or GA). As described in Examples 5 and 6 below, low doses of Hsp90 inhibitors (e.g., 17-AAG) having a minimal impact on cell growth, cause a robust decrease in AID activities. More importantly, as presented in Example 7 below, a low dose of Hsp90 inhibitors can prevent, in the CML cell line K562, the AID-driven generation of imatinib resistance. Hsp90 inhibitors could thus be used to inhibit AID expression and/or activity in the treatment of human diseases.

AID expression and/or activity in a human cancer is indicative that the cancer may progress and is highly susceptible to develop resistance to drug treatment.

Therefore, in an aspect, the present invention provides a pharmacological method to reduce AID expression and/or activity. The present invention also provides a method for assessing AID expression and/or activity in samples of subjects having or likely to develop an AID-associated disease to determine whether or not a treatment inhibiting AID is appropriate.

In one embodiment of the present invention, the presence of AID in association with a tissue (e.g., neoplastic or pre neoplastic tissues, population of B cells) is used for subject stratification. The level of AID expression and/or activity is used to decide whether or not a treatment with a Hsp90 inhibitor (e.g., 17-AAG) is appropriate and to which dose and length of treatment. It is thus possible to decrease certain side effects of a treatment (e.g., liver toxicity) by selecting the effective dose of inhibitory compound having an effect on AID expression and/or activity. In a more specific embodiment, subject stratification is further performed by detecting other relevant clinical factors such as hyperplasia or other relevant premalignant lesions, or a decreased expression and/or activity of a gene affecting AID mutator activity by controlling or repairing DNA damage (e.g., p53 loss-of-function mutations).

In another aspect, the present invention provides a method for treating a cancer, preventing cancer progression and/or development of drug resistance in a subject comprising measuring AID expression and/or activity in a sample from the subject and wherein if AID expression and/or activity is detected, an effective amount of a Hsp90 inhibitor (i.e., an agent capable of inhibiting AID expression and/or activity) is administered to the subject. In a specific embodiment, the Hsp90 inhibitor is administered in combination with at least one other therapeutic agent (e.g., 17-AAG combined to imatinib in AID-positive CML).

In another embodiment of the present invention, the treatment is a monotherapy using an inhibitor of AID. In one embodiment, the monotherapy treatment is directed to the prevention of cancer development in a patient having an AID positive pre neoplastic tissue.

In another embodiment of the present invention, the treatment is directed to the treatment and prevention of autoimmune diseases in a patient having an aberrant AID activity in a B cell tissue.

In one aspect, the invention provides a method for adjusting a dose in a Hsp90 inhibitor treatment, comprising measuring the level of AID expression and/or activity in a biological sample of a patient under treatment with an Hsp90 inhibitor and administering to patient having aberrant AID expression and/or activity an increased dose of said Hsp90 inhibitor.

In one embodiment, the treatment administering an Hsp90 inhibitor (e.g., 17-AAG) is combined to a treatment (e.g., administration of estrogen, administration of proinflammatory cytokine) known to increase AID expression and/or activity.

In one embodiment, the treatment administering an Hsp90 inhibitor (e.g., 17-AAG) is directed to the treatment of allergy.

In another aspect, the present invention provides a Hsp90 inhibitor, or a composition comprising said inhibitor, and a pharmaceutically acceptable carrier, for preventing and/or treating a subject having a tumor expressing AID.

### AID Gene and AID Protein

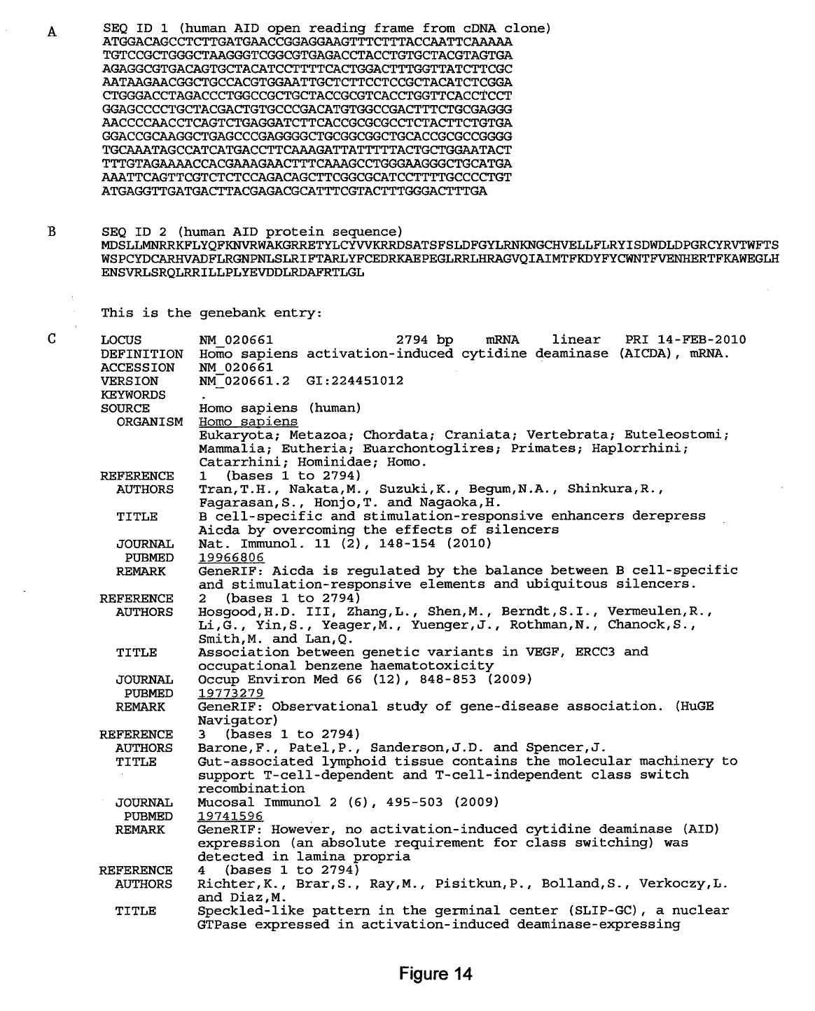

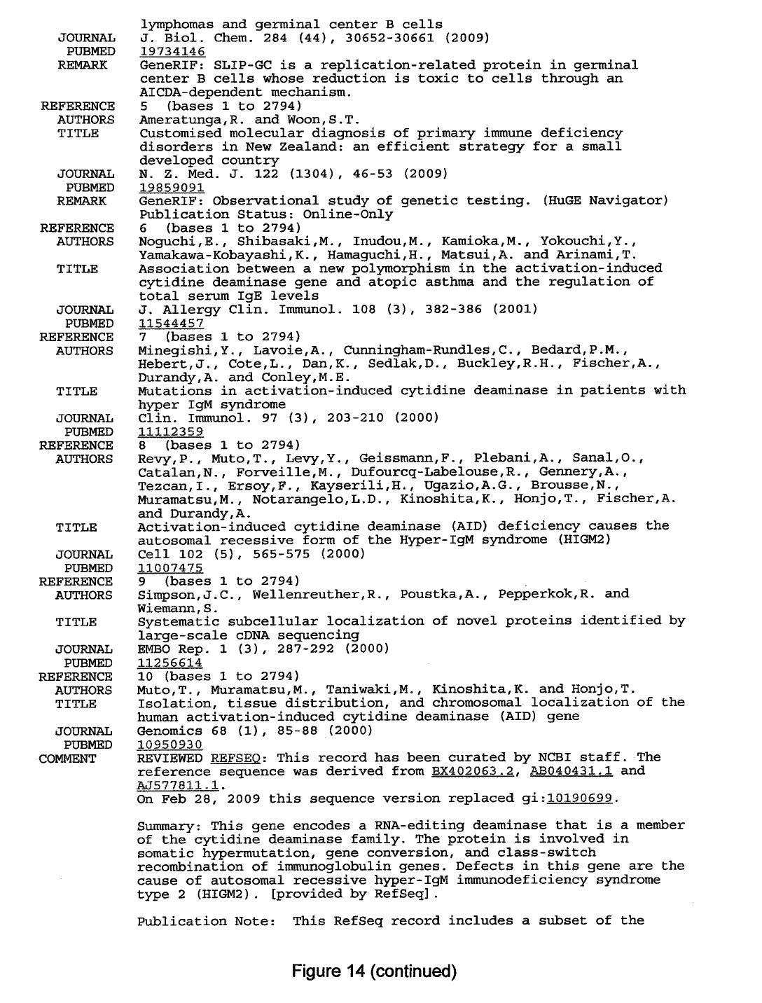

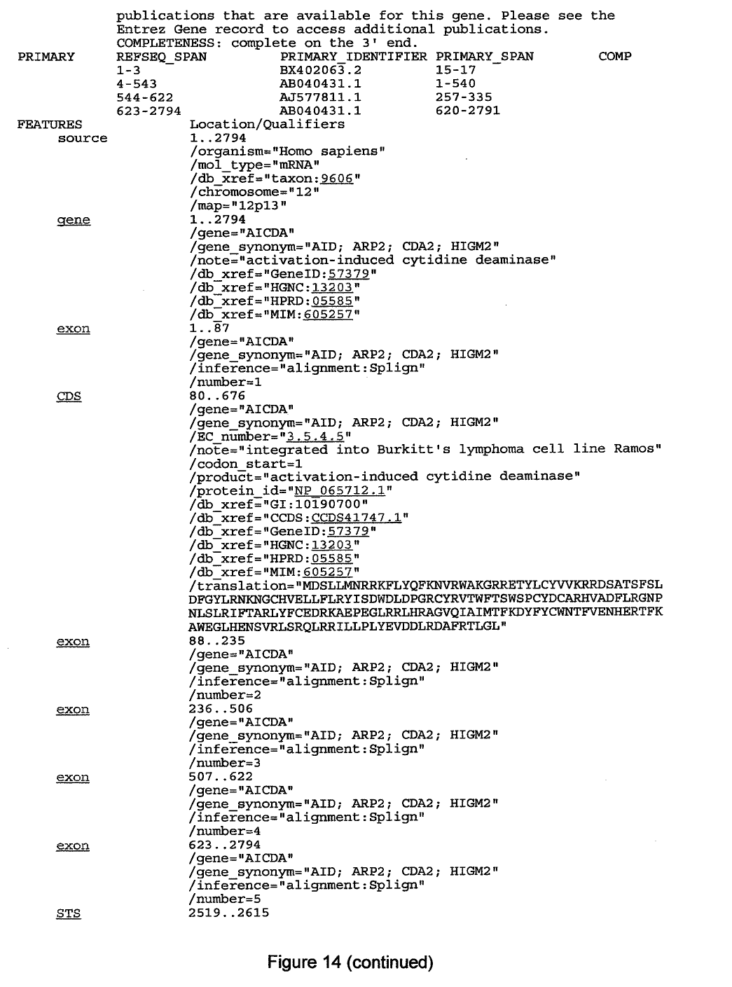

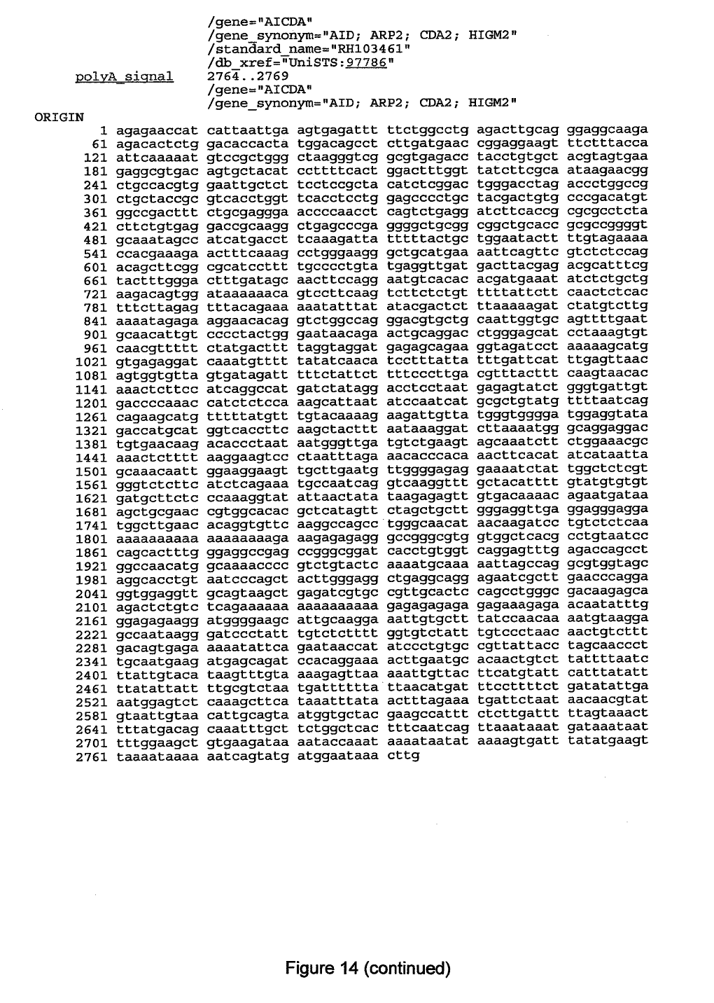

As used herein the terms “AID gene” refers to nucleic acid (e.g., genomic DNA, cDNA, RNA) encoding Activation Induced Deaminase (AID) (e.g., sequences comprising those sequences referred to in GenBank by accession number NM_{—}020661 and NG_{—}011588 for the human gene. Although the term AICDA is typically used when designating the gene encoding AID, the expression “AID gene” will be used herein for convenience and consistency. The description of the various aspects and embodiments of the invention is provided with reference to exemplary AID nucleic acid sequences (SEQ ID NOs: 1 and 3) and amino acid sequence (SEQ ID NO: 2) (FIG. 14). Such reference is meant to be exemplary only and the various aspects and embodiments of the invention are also directed to other AID nucleic acids and polypeptides (also referred to AID gene products), such as AID nucleic acid or polypeptide mutants/variants, splice variants of AID nucleic acids, AID variants from species to species or subject to subject. Without being so limited, those include AID sequences at accession numbers NG_{—}011588 Homo sapiens activation-induced cytidine deaminase (AICDA) on chromosome 12 gi|224994215|ref|NG_{—}011588.1| [224994215; NC_{—}000012 Homo sapiens chromosome 12, GRCh37 primary reference assembly gi|224589803|ref|NC_{—}000012.11∥gpp|GPC_{—}000000036.1∥gnl|NCBI_GENOMES|12 [224589803; NT_{—}009714 Homo sapiens chromosome 12 genomic contig, GRCh37 reference primary assembly gi|224514867|ref|NT_{—}009714.17∥gpp|GPS_{—}000125290.1| [224514867]; NM_{—}020661 Homo sapiens activation-induced cytidine deaminase (AICDA), mRNA gi|224451012|ref|NM_{—}020661.2| [224451012]; 5: AC_{—}000144 Homo sapiens chromosome 12, alternate assembly HuRef, whole genome shotgun sequence gi|157704453|ref|AC_{—}000144.1∥gnl|NCBI_GENOMES|21406 [157704453]; NW_{—}001838051 Homo sapiens chromosome 12 genomic contig, alternate assembly (based on HuRef), whole genome shotgun sequence gi|157696928|ref|NW_{—}001838051.1| [157696928]; DQ896237 Synthetic construct Homo sapiens clone IMAGE:100010697; FLH191441.01L; RZPDo839D0467D activation-induced cytidine deaminase (AICDA) gene, encodes complete protein gi|123999319|gb|DQ896237.2| [123999319]; DQ892989 Synthetic construct clone IMAGE: 100005619; FLH191445.01X; RZPDo839D0477D activation-induced cytidine deaminase (AICDA) gene, encodes complete protein gi|123990479|gb|D0892989.2| [123990479]; AM393608 Synthetic construct Homo sapiens clone IMAGE:100002005 for hypothetical protein (AICDA gene)gi|117646033|emb|AM393608.1|[117646033]; D0431660 Homo sapiens activation-induced cytidine deaminase mRNA, partial cds gi|90200384|gb|D0431660.1| [90200384]; AC_{—}000055 Homo sapiens chromosome 12, alternate assembly Celera, whole genome shotgun sequence gi|89161189|ref|AC_{—}000055.1∥gnl|NCBI_GENOMES|18894 [89161189]NW_{—}925295 Homo sapiens chromosome 12 genomic contig, alternate assembly (based on Celera), whole genome shotgun sequence gi|89035948|ref|NW_{—}925295.1| [89035948]; CH471116 Homo sapiens 211000035838052 genomic scaffold, whole genome shotgun sequence gi|74230026|gnl|WGS:AADB|211000035838052|gb|CH471116.2| [74230026]; CS056120 Sequence 39 from Patent WO2005023865 gi|162122322|emb|CS056120.1∥pat|WO12005023865|39 [62122322]; AY748364 Homo sapiens activation-induced deaminase (AICDA) mRNA, partial cds gi|53854919|gb|AY748364.1| [53854919]; CR615215 full-length cDNA clone CS0DL012YD18 of B cells (Ramos cell line) Cot 25-normalized of Homo sapiens (human)gi|50496022|emb|CR615215.1| [50496022]; AY541058 Homo sapiens activation-induced cytidine deaminase (AICDA) mRNA, complete cds, alternatively spliced gi|46484694|gb|AY541058.1| [46484694]; AY536517 Homo sapiens activation-induced cytidine deaminase (AICDA) mRNA, complete cds, alternatively spliced gi|46403718|gb|AY536517.1| [46403718]; AY536516 Homo sapiens activation-induced cytidine deaminase (AICDA) mRNA, complete cds, alternatively spliced gi|46403716|gb|AY536516.11 [46403716]; AY534975 Homo sapiens activation-induced cytidine deaminase (AICDA) mRNA, complete cds, alternatively spliced gi|46371948|gb|AY534975.1| [46371948]; BC006296 Homo sapiens activation-induced cytidine deaminase, mRNA (cDNA clone MGC:12911 IMAGE:4054915), complete cds gi|33871601|gb|BC006296.21 [33871601]; AJ577811 Homo sapiens partial mRNA for activation-induced cytidine deaminase (AID gene) gi|33145978|emb|AJ577811.1| [33145978]; BT007402 Homo sapiens activation-induced cytidine deaminase mRNA, complete cds gi|30583642|gnl|clontech|GH00009X1.0|gb|BT007402.1| [30583642]; AB092577 Homo sapiens AID gene for activation-induced cytidine deaminase, partial cds, exon 2 gi|291260421 dbj|AB092577.1| [29126042]; AF529827 Homo sapiens clone Ramos 13 AID (AID) mRNA, partial cds gi|22297241|gb|AF529827.1| [22297241]; AF529826 Homo sapiens clone Ramos 12 AID (AID) mRNA, partial cds gi|22297239|gb|AF529826.1| [22297239]; AF529825 Homo sapiens clone Ramos 11 AID (AID) mRNA, partial cds gi|22297237|gb|AF529825.1| [22297237]; AF529824 Homo sapiens clone Ramos 10 AID (AID) mRNA, partial cds gi|22297235|gb|AF529824.1| [22297235]; AF529823 Homo sapiens clone Ramos 9 AID (AID) mRNA, partial cds gi|22297233|gb|AF529823.1| [22297233]; AF529822 Homo sapiens clone Ramos 8 AID (AID) mRNA, partial cds gi|22297231|gb|AF529822.1| [22297231]; AF529821 Homo sapiens clone Ramos 7 AID (AID) mRNA, partial cds gi|22297229|gb|AF529821.1| [22297229]; AF529820 Homo sapiens clone Ramos 6 AID (AID) mRNA, partial cds gi|22297227|gb|AF529820.1| [22297227]; AF529819 Homo sapiens clone Ramos 5 AID (AID) mRNA, partial cds gi|22297225|gb|AF529819.1| [22297225]; AF529818 Homo sapiens clone Ramos 4 truncated AID (AID) mRNA, complete cds gi|22297223|gb|AF529818.1| [22297223]; AF529817 Homo sapiens clone Ramos 3 AID (AID) mRNA, partial cds gi|22297221|gb|AF529817.1| [22297221]; AF529816 Homo sapiens clone Ramos 2 AID (AID) mRNA, partial cds gi|22297219|gb|AF529816.1| [22297219]; AF529815 Homo sapiens clone Ramos 1 AID (AID) mRNA, partial cds gi|22297217|gb|AF529815.1| [22297217]; AC092184 Homo sapiens 12 BAC RP11-438L7 (Roswell Park Cancer Institute Human BAC Library) complete sequence gi|21206067|gnl|bcmhgsc|project_hdkj.baylor|gb|AC092184.71 [21206067]; AB040431 Homo sapiens AID mRNA for activation-induced cytidine deaminase, complete CDS gi|9988409|dbj|AB040431.1| [9988409]; AB040430 Homo sapiens AID gene for activation-induced cytidine deaminase, complete cds gi|9988407|dbj|AB040430.1| [9988407]. Without being so limited, examples of mutant variants are described in ^{21,57}.

### AID Expression

As used herein the terms “AID expression level” or “AID expression” refer to the measurement in a cell or a tissue of an AID gene product. AID expression levels could be evaluated at the polypeptide and/or nucleic acid levels (e.g., DNA or RNA) using any standard methods known in the art. The nucleic acid sequence of a nucleic acid molecule in a sample can be detected by any suitable method or technique of measuring or detecting gene sequence or expression. Such methods include, but are not limited to, polymerase chain reaction (PCR), reverse transcriptase-PCR (RT-PCR), in situ PCR, SAGE, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene.

In the context of this invention, “hybridization” means hydrogen bonding between complementary nucleoside or nucleotide bases. Terms “specifically hybridizable” and “complementary” are the terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. Such conditions may comprise, for example, 400 mM NaCl, 40 mM PIPES pH 6.4, 1 mM EDTA, at 50 to 70° C. for 12 to 16 hours, followed by washing. The skilled person will be able to determine the set of conditions most appropriate for a test of complementarity of two sequences in accordance with the ultimate application of the hybridized nucleotides.

Methods to measure protein expression levels of selected genes of this invention, include, but are not limited to: Western blot, tissue microarray, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), flow cytometry, and assays based on a property of the protein including but not limited to DNA binding, ligand binding, or interaction with other protein partners. In a further embodiment, the AID expression level is measured by immunohistochemical staining, and the percentage and/or the intensity of immunostaining of immunoreactive cells in the sample is determined.

In an embodiment, the level of an AID polypeptide is determined using an anti-AID antibody. By “AID antibody” or “anti-AID” in the present context is meant an antibody capable of detecting (i.e. binding to) an AID protein or an AID protein fragment. Without being limited, AID antibodies includes those listed in Table I below.

TABLE I
Examples of commercially available AID antibodies

| Company | Catalog number | Name |
|---|---|---|
| Cell signaling technologies | 4959 | EK2 5G9 Rat mAb |
| | 4975 | L7E7 Mouse mAb |
| | 30F12 | Rabbit mAb |
| Abcam | Ab5197 | Rabbit polyclonal |
| | Ab59361 | Rabbit polyclonal |
| | Ab77401 | Goat polyclonal |
| | Ab56147 | Rabbit polyclonal |
| Genway | 18-202-336474 | Rabbit polyclonal |
| | 18-783-313040 | Rabbit polyclonal |

Methods for normalizing the level of expression of a gene are well known in the art. For example, the expression level of a gene of the present invention can be normalized on the basis of the relative ratio of the mRNA level of this gene to the mRNA level of a housekeeping gene, or the relative ratio of the protein level of the protein encoded by this gene to the protein level of the housekeeping protein, so that variations in the sample extraction efficiency among cells or tissues are reduced in the evaluation of the gene expression level. A “housekeeping gene” is a gene the expression of which is substantially the same from sample to sample or from tissue to tissue, or one that is relatively refractory to change in response to external stimuli. A housekeeping gene can be any RNA molecule other than that encoded by the gene of interest that will allow normalization of sample RNA or any other marker that can be used to normalize for the amount of total RNA added to each reaction. For example, the GAPDH gene, the G6PD gene, the actin gene, ribosomal RNA, 36B4 RNA, PGK1, RPLP0, or the like, may be used as a housekeeping gene.

Methods for calibrating the level of expression of a gene are well known in the art. For example, the expression of a gene can be calibrated using reference samples, which are commercially available. Examples of reference samples include, but are not limited to: Stratagene™ QPCR Human Reference Total RNA, Clontech™ Universal Reference Total RNA, and XpressRef™ Universal Reference Total RNA.

In an embodiment, the above-mentioned method comprises determining the level of an AID nucleic acid (e.g., the nucleic acid of SEQ ID NO: 1) in the sample. In another embodiment, the above-mentioned method comprises determining the level of an AID polypeptide (e.g., the polypeptide of SEQ ID NO: 2) in the sample.

### AID Activity

As used herein the terms “AID activity” and “AID function” are used interchangeably and refer to detectable (direct or indirect) enzymatic (e.g., deamination of deoxycytidine (dC) to deoxyuridine (dU)), biochemical or cellular activity attributable to AID. Without being so limited, such activities include the binding of AID to Hsp90, the binding of AID to CHIP, the effect of AID on cellular genomic plasticity such as a dU-induced DNA break, a DNA translocation, a DNA deletion, a DNA recombination (including region-specific recombination between isotype switch regions, immunoglobulin gene conversion, homologous recombination) or a general or localized mutator effect. Other activities of AID include Ig gene (i.e. encoding antibody) diversification by somatic hypermutation (SHM) and class switch recombination (CSR) (e.g., IgM to IgG, IgE or IgA). Assays measuring SHM and CSR are described in Example 1 below and results of these assays are presented in Examples 5 and 6 for example. AID activity could also be indirectly measured by evaluating the level of expression of AID, or a fragment thereof, in cells as well as in biological samples (e.g., tissue, organ, fluid).

### Modulation of AID Expression or Activity

The modulation of AID expression and/or activity could be achieved directly or indirectly by various mechanisms, which among others could act at the level of (i) transcription, for example by stimulating the AID promoter increasing the AID messenger RNA expression (e.g., by cytokine stimulation, Toll-like receptor stimulation, estrogen-estrogen receptor complex, HCV core protein, EBV LMP2, etc.), (ii) translation, (iii) post-translational modifications, e.g., glycosylation, sulfation, phosphorylation, ubiquitination (e.g., polyubiquitinylation and proteasomal degradation), (iv) cellular localization (e.g., cytoplasmic versus nuclear localization), (v) protein-protein interaction, for example by modulating expression and/or activity of a protein that binds to and stabilizes AID (e.g., Hsp90 as well as other members of the Hsp90 chaperoning pathway including the Hsp40 cochaperones DnaJa1 and DnaJa2, AHA-1, BAG-2, the Hsp90-associated ubiquitin ligase CHIP, the so far uncharacterized pathway destabilizing AID in the nucleus⁶⁰. These regulatory processes occur through different molecular interactions that could be modulated using a variety of compounds or modulators.

An important step regulating AID is subcellular localization. Most of the enzyme is in the cytoplasm in steady state, which is determined by the integration of three mechanisms: nuclear import²⁸, nuclear export^{58,59} and cytoplasmic retention²⁸. The compartmentalization of AID determines its stability: AID is destabilized in the nucleus by polyubiquitinylation and proteasomal degradation 60

As indicated above, modulation of AID mutator activity can also be achieved by the activity resulting from genes known to control/prevent/repair AID-mediated mutations and/or AID-mediated antibody diversification. These include, amongst others, p53, ATM, Nbs1, p19(Arf), PUMA, Bim, PKCdelta and UNG2.

In the context of the present invention, a “compound” is a molecule such as, without being so limited, siRNA, antisense molecule, protein, peptide, small molecule, antibodies, etc.

### AID as a New Hsp90 Client Protein

Hsp90 is a protein chaperone that binds to several sets of signaling proteins, known as “client proteins”. Hsp90 is thought to be more selective of its range of substrates than other chaperones, playing a more prominent role in the structural stabilization and functional modulation of many of its client proteins, rather than in their initial folding (reviewed in ⁶¹⁻⁶⁶). These client proteins include a “who's who” list of cancer-relevant targets such as mutated (but not normal) p53, Bcr-Abl1, Raf-1, ErbB2, Her2, Akt, c-Raf, Cdk4, Cyclin D1 as well as other kinases and steroid hormone receptors. AID is a novel client for Hsp90. Disruption of the Hsp90-client protein complexes leads to proteosome-mediated degradation of client proteins. The binding of Hsp90 to a client protein is dependent on the ATPase activity of Hsp90.

### Hsp90 Inhibitors

In the context of the present invention, the term “Hsp90 inhibitor” includes any compound able to directly or indirectly affect the ability of Hsp90 to bind to and/or stabilize AID. One class of Hsp90 inhibitors includes molecules that inhibit the ATPase activity of Hsp90 by interacting with the ATP binding pocket in the N-terminal domain. Another class of Hsp90 inhibitors interacts with the C-terminal domain of Hsp90.

In the context of the present invention, examples of Hsp90 inhibitors include the benzoquinone ansamycin geldanamycin and analogs thereof such as the 17-(Allylamino)-17-demethoxygeldanamycin (17-MG, Tanespimycin, Retaspimycin hydrochloride), 17-(Dimethylaminoethylamino)-17-demethoxygeldanamycin (17-DMAG, Alvespimycin), nab-17-AAGs (e.g., ABI-010, Abraxis BioScience Inc); lipid formulation of ansamycin-based Hsp90 modulators (e.g. CNF1010, Biogen); macrolides, for example, Pochonin, Radester and Radicicol-based Hsp90 inhibitors (e.g., NXD30001, NexGenix Pharmaceuticals); the purine-scaffold derivatives, for example, PU-3, PUFCI, AT-13387 and 8-arylsulfanyl adenine derivatives such as 8-(2-iodo-5-methoxy-phenylsulfanyl)-9-pent-4-ynyl-9H-purin-6-ylamine; other known Hsp90 inhibitors such as Herbimycin, Shepardin, Cisplatin, aminocoumarin antibiotic Novobiocin and the Novobiocin-derived KU135 and F-4 a^{67,68}; pyrazoles such as CCT018159 (4-[4-(2,3-Dihydro-1,4-benzodioxin-6-yl)-5-methyl-1H-pyrazol-3-yl]-6-ethyl-l-1,3-benzenediol); and BTIMNP_D004, a natural plant extract that reduces the Hsp90 expression⁶⁷. Without being so limited, further HSP90 inhibitors encompassed by the present invention are described in US2008000023202; US20080153837A1; US2006000541462; EP2036895A1; WO2009007399A1; EP2065388A1; WO120091097578; US20100022635.

Both benzoquinone ansamycins and radicicol-based hsp90 inhibitors act on the ATPase activity of Hsp90 (N-terminal), Novobiocin and cisplatin interact with the C-terminal domain of Hsp90 and have a different mechanism of inhibition. Please also see Table II below listing Hsp90 inhibitors.

TABLE II
HSP90 inhibitors

| | | Compounds/ | | | |
|---|---|---|---|---|---|
| | | Drug names | | developmental | Company |
| Chemical class | Lead compound | Trade names | Route | status | name |
| Natural | Radicicol-based | NXD30001 | | | NexGenix |
| antibiotic- | Benzochinone | 17-AAG | Iv | Phase II | |
| based HSP90- | Ansamycins | (NCI-formulation) | | | |
| Inhibitors | | 17-AAG | Iv | Phase II | Kosan |
| | | (cremaphor and | | | |
| | | suspension | | | |
| | | formulation) | | | |
| | | Tanespimycin (KOS- | | | |
| | | 953) | | | |
| | | IPI-504 | iv | Phase III in GIST | Infinity |
| | | Retaspimycin | | | |
| | | IPI-493 | Oral | Phase I | Infinity |
| | | 17-DMAG | Iv and | Phase II/III | Kosan |
| | | KOS-1022, | oral | | |
| | | Alvespimycin, | | | |
| | | CNF-1010 (oil in | Iv | Phase I/II | Biogen |
| | | water emulsion) | | | |
| | | Macbecin | n.k. | preclinical | Biotica |
| Pyrazoles | Resorcinol | CCT018159 | | | |
| | | VER-49009 (CCT- | | | |
| | | 129397) and others | | | |
| | | BIIB021 (CNF 2024) | oral | Phase I/II in | Biogen- |
| | | | | GIST | Idec |
| Purine-based | | AT-13387 | n.k. | Phase I in solid | Astex |
| | | | | tumors | |
| Other small | | PF-04928473 | oral | Phase I in solid | Pfizer |
| molecule | | | | tumors | |
| inhibitors | | | | | |
| | | STA9090 | Oral | Phase I, solid | Synta |
| | | | | tumors | |
| | | AUY922 | | Phase I, solid | Novartis |
| | | | | tumors | |
| | | MPC-3100 | Oral | Phase I | Myriad |
| | | CUDC-305 | Oral | | Curis |
| | | XL888 | Oral | | Exelixis |

### AID Inhibitors

As used herein, “AID inhibitor” refers to any compound or composition that directly or indirectly inhibits AID expression and/or activity. In the context of the present invention, Hsp90 inhibitors are one class of AID inhibitors. Without being so limited, candidate compounds modulating the AID expression and/or activity are tested using a variety of methods and assays some of which are described in Examples 3, 4 (for AID expression); and 5-7 (for AID activities).

As used herein, “inhibition” or “decrease” of AID expression and/or activity refers to a reduction in AID expression level or activity level of at least 5% as compared to reference AID expression and/or activity (e.g., a measurement of AID expression and/or activity in the subject before treatment with an Hsp90 inhibitor). In an embodiment, the reduction in AID expression level or activity level is of at least 10% lower, in a further embodiment, at least 15% lower, in a further embodiment, at least 20% lower, in a further embodiment of at least 30%, in a further embodiment of at least 40%, in a further embodiment of at least 50%, in a further embodiment of at least 60%, in a further embodiment of at least 70%, in a further embodiment of at least 80%, in a further embodiment of at least 90%, in a further embodiment of 100% (complete inhibition).

Preferably, an AID inhibitor is a compound having a low level of cellular toxicity and acting in a reversible manner.

### AID-Associated Diseases

As used herein the terminology “AID-associated diseases” includes, without being so limited, AID-expressing neoplastic diseases including AID-expressing solid tumors (e.g., inflammation-associated cancers) and AID-expressing immune system-derived cancers, and other immune system diseases including atopic allergies and B cell-related autoimmune diseases (e.g., systemic lupus erythematosus).

Among AID-associated diseases certain are estrogen-driven (e.g., caused by treatment with estrogen) including certain AID-expressing neoplastic diseases such as certain breast and ovarian cancer, and certain B cell-related autoimmune diseases such as Rheumatoid Arthritis, System Lupus Erythematosus and Multiple Sclerosis.

“AID-expressing immune system-derived cancers” include herein but are not restricted to, chronic myeloid leukemia (CML)²³; acute lymphoblastic leukemia (e.g., BCR-ABL1-positive ALL)²²; human B cell non-Hodgkin's lymphomas (B-NHLs), such as follicular lymphoma (FL)^{19,20,33,69}, Burkitt lymphoma^{19,20}, all subtypes of diffuse large B-cell lymphoma (DLBCL)^{19,20,44,69} and AIDS-associated B-NHL⁵⁴ as well as in B-cell chronic lymphocytic leukemia (B-CLL), and its tissue counterpart, small lymphocytic lymphoma (SLL)^{33,70}.

“AID-expressing solid tumors” include herein but are not restricted to, stomach tumor (e.g., Helicobacter pylori infection-associated stomach tumor), gastric adenocarcinomas³¹, cholangiocarcinoma³², lymph node lymphomas^{19,20,44,69}, lung tumor¹⁸, liver tumor⁷¹, colitis-associated colorectal cancers²⁴, brain tumor, ovary tumor (e.g., ovary carcinoma, endometriosis or adenocarcinoma), breast tumor⁷² (e.g., breast fibroadenoma or carcinoma), skin tumor (e.g., skin melanoma), prostate carcinoma, bladder tumor (e.g., bladder adenocarcinoma), vascular endothelium hemangioma, kidney carcinoma, thyroid follicular adenoma, relapsed-refractory multiple myeloma. Several data strongly suggest the involvement of AID in inflammation-associated carcinogenesis in humans³⁴. For instance, aberrant AID expression was revealed in colonic mucosa and cancer tissues of patient with inflammatory bowel disease, but not in normal colonic mucosa.

In one embodiment, the present invention relates to benign neoplastic disease. In another embodiment the present invention relates to malignant neoplastic disease. In specific embodiments, the malignant neoplastic disease is cancer.

In an embodiment, the above-mentioned cancer/tumor is associated with AID expression and/or activity (e.g., aberrant or increased AID expression and/or activity, also referred to as AID-expressing or AID-positive tumor). In one embodiment, the above-mentioned cancer is a cancer of the immune system.

In another embodiment, the above-mentioned cancer/tumor is a solid tumor.

### Clinical Applications of Hsp90 Inhibitors in Cancer Treatment

Because the Hsp90 client proteins are so important in signal transduction and in transcription, geldanamycin analogs such as 17-AAG serve as chemotherapeutic agents in a number of cancers. An overview of important pre-clinical development data (see Table II above) is provided by Porter et al.⁷³. Preclinical studies suggest that these compounds are synergistic with certain other inhibitors of the signal transduction client proteins, as well as with several conventional anticancer agents.

Hsp90 inhibitors are being developed for the treatment of a variety of cancers including solid tumors (e.g., thyroid cancer, HER-2 positive metastatic breast cancer, kidney cancer, metastatic melanoma) as well as lymphoma, CML and relapsed-refractory multiple myeloma. 17-AAG harbors anti cancer activities and is involved in several clinical trials (phase I, II and III; 2002, 2008, 2009, also reviewed in ⁷⁴). Not surprisingly however, according to the central role of Hsp90 in various cellular processes, a number of dose-limiting toxicities for Hsp90 inhibitors have been identified (e.g., for 17-AAG in ⁷⁵).

Because Hsp90 inhibitors affect AID expression and/or activity, one possible adverse effect of treating cancer with Hsp90 inhibitors would be a reduction of the normal AID activities such as the reduction of somatic hypermutation and class switch recombination in normal B cells.

A sustained treatment with Hsp90 inhibitors may have some negative effect on antibody-mediated immune responses. This should have a relatively minor impact on the health of immunocompetent adults and little effect on any cell-mediated anti tumoral immune responses. Nevertheless, some effects on cellular immunity (i.e., T-cell, NK-cell mediated) might be possible through the known effect of Hsp90 on important signaling molecules in various immune cells. The actual effect is likely to vary and depend on the pharmacokinetic characteristics of each particular Hsp90 inhibitor.

In addition to their toxicity, the potency, tolerability, pharmacokinetic and pharmacodynamic properties of the known Hsp90 inhibitors also differ. For instance, results indicate that NXD30001 and its derivatives may be useful in the treatment of breast cancer with an improved dosing and therapeutic window compared to the most extensively studied and validated Hsp90 inhibitors, geldanamycin-based 17-AAG. NXD30001 has shown enhanced Hsp90 binding affinity, and potency in inhibiting cell growth in vitro in various cancer cell lines compared to 17-MG and 17-DMAG. CNF1010 is a lipid formulation of a semi-synthetic analogue of geldanamycin with improved pharmaceutical properties. Such compound has a striking ability to induce degradation of signaling molecules, including HER2/neu.

### Treatment and Prevention

The terms “treat/treating/treatment” and “prevent/preventing/prevention” as used herein, refers to eliciting the desired biological response, i.e., a therapeutic and prophylactic effect, respectively. In accordance with the subject invention, the therapeutic effect comprises one or more of a decrease/reduction in the severity of a human diseases (e.g., a reduction or inhibition of cancer progression and/or metastasis development or reduction or inhibition of an autoimmune disease), a decrease/reduction in symptoms and disease-related effects, an amelioration of symptoms and disease-related effects, a decrease/reduction of the development of the cancer resistance to a drug treatment, and an increased survival time of the affected host animal, following administration of the at least one Hsp90 inhibitor (or of a composition comprising the inhibitor). In accordance with the invention, a prophylactic effect may comprise a complete or partial avoidance/inhibition or a delay of cancer (e.g., a complete or partial avoidance/inhibition or a delay of metastasis development), of drug resistance, or of autoimmune disease development/progression, and an increased survival time of the affected host animal, following administration of the at least one Hsp90 inhibitor (or of a composition comprising the inhibitor).

As such, a “therapeutically effective” or “prophylactically effective” amount of Hsp90 inhibitors affecting AID expression and/or activity, or a combination of such inhibitors, may be administered to an animal, in the context of the methods of treatment and prevention, respectively, described herein.

Types of Samples from the Subject and of Control Samples

As used herein, the term “organism” refers to a living thing which, in at least some form, is capable of responding to stimuli, reproduction, growth or development, or maintenance of homeostasis as a stable whole (e.g., an animal). The organism may be composed of many cells which may be grouped into specialized tissues or organs.

“Sample” or “biological sample” refers to any solid or liquid sample isolated from a live being. In a particular embodiment, it refers to any solid (e.g., tissue sample) or liquid sample isolated from a human, such as a biopsy material (e.g., solid tissue sample), blood (e.g., plasma, serum or whole blood), saliva, synovial fluid, urine, amniotic fluid and cerebrospinal fluid. Such sample may be, for example, fresh, fixed (e.g., formalin-, alcohol- or acetone-fixed), paraffin-embedded or frozen prior to analysis of AID expression level. In an embodiment, the above-mentioned sample is obtained from a tumor.

As used herein, the term “tissue” or “tissue sample” refers to a group of cells, not necessarily identical, but from the same origin, that together carry out a specific function. A tissue is a cellular organizational level intermediate between cells and a complete organism. Organs are formed by the functional grouping together of multiple tissues. Examples of tissues include dermal, adipose, connective tissue, epithelial, muscle, nervous tissues. Other examples of biological tissues include blood cells populations (e.g., B or T lymphocytes populations), breast or ovarian tissues.

The expression “reference AID expression and/or activity” refers to the AID expression and/or activity used as a control for the measure performed in a sample from a subject. “Reference AID sample” as used herein refers to a sample comprising a reference AID expression and/or activity.

Depending on the type of assay performed, the reference AID expression and/or activity can be selected from an established standard, a corresponding AID expression and/or activity determined in the subject (in a sample from the subject) at an earlier time; a corresponding AID expression and/or activity determined in one or more control subject(s) known to not being predisposed to an AID-associated disease, known to not having an AID-associated disease, or known to have a good prognosis; known to have a predisposition to an AID-associated disease or known to have an AID-associated disease (e.g., a specific tumor subtype) or known to have a poor prognosis. In another embodiment, the reference AID expression and/or activity is the average or median value obtained following determination of AID expression or activity in a plurality of samples (e.g., samples obtained from several healthy subjects or samples obtained from several subjects having an AID-associated disease (e.g., cancer)).

Similarly, the expression “reference expression and/or activity of a gene” refers to the expression and/or activity of that gene used as a control for the measure performed in a sample from a subject. “Reference sample of a gene” as used herein refers to a sample comprising a reference expression and/or activity of a gene.

Similarly, the reference expression and/or activity of a gene known to regulate AID mutator activity by controlling or repairing DNA damage can be selected from an established standard, a corresponding expression and/or activity determined in one or more control subject(s) known to not being predisposed to an AID-associated disease, known to not having an AID-associated disease, or known to have a good prognosis; known to have a predisposition to an AID-associated disease or known to have an AID-associated disease or known to have a poor prognosis. In another embodiment, the reference expression and/or activity of a gene known to regulate AID mutator activity by controlling or repairing DNA damage is the average or median value obtained following determination of expression or activity of the gene known to regulate AID mutator activity by controlling or repairing DNA damage in a plurality of samples (e.g., samples obtained from several healthy subjects or samples obtained from several subjects having an AID-associated disease (e.g., cancer)).

“Corresponding normal tissue” or “corresponding tissue” as used herein refers to a reference sample obtained from the same tissue as that obtained from a subject. Corresponding tissues between organisms (e.g., human subjects) are thus tissues derived from the same origin (e.g., two ovarian tissues, two B lymphocyte populations).

### Measurement of AID in a Sample

The present invention encompasses methods comprising determining whether AID activity and/or expression in a subject sample is higher than a reference expression and/or activity.

The present invention also encompasses method comprising determining whether AID expression in B cells of a subject sample is substantially similar to a reference expression but in the context of an independent predisposing condition (e.g., (a) a reduced capacity for controlling/preventing/repairing DNA damage; and/or (b) a deficiency in specific DNA repair enzymes known to repair uracil in DNA) which results from a genetic mutation leading to an increase of the mutator activity of AID in the B cells (e.g., a loss-of-function mutation in TP53, ATM, or UNG2).

In cases where the reference AID sample is from the subject at an earlier time; from subject(s) known not to being predisposed to an AID-associated disease, known not to have an AID-associated disease, or known to have a good prognosis, an increased/higher AID expression and/or activity in the sample from the subject relative to the reference AID expression and/or activity is indicative that the subject has an AID-associated disease, has a predisposition to an AID-associated disease (e.g., has a higher risk of developing an AID-associated disease and/or of experiencing an AID-associated disease progression) or has a poor prognosis (e.g., lower survival probability, higher probability of AID-associated disease recurrence), while a comparable or lower expression or activity in a sample from the subject relative to the reference expression and/or activity is indicative that the subject does not have an AID-associated disease, is not predisposed to an AID-associated disease or has a good prognosis (e.g., higher survival probability, lower probability of cancer recurrence).

In cases where the reference AID sample is from subject(s) known to have a predisposition to an AID-associated disease, known to have an AID-associated disease or known to have a poor prognosis, a comparable or increased/higher AID expression and/or activity in a sample from the subject relative to the reference AID expression and/or activity is indicative that the subject has an AID-associated disease, has a predisposition to an AID-associated disease or has a poor prognosis (e.g., lower survival probability, higher probability of AID-associated disease recurrence), while a lower expression or activity in a sample from the subject relative to the reference expression and/or activity is indicative that the subject does not have an AID-associated disease, is not predisposed to an AID-associated disease or has a good prognosis (e.g., higher survival probability, lower probability of AID-associated disease recurrence).

As used herein, a “higher” or “increased” level refers to levels of expression or activity in a sample (i.e. sample from the subject) which exceeds with statistical significance that in the reference sample (e.g., an average corresponding level of expression or activity a healthy subject or of a population of healthy subjects, or when available, the normal counterpart of the affected or pathological tissue) measured through direct (e.g., Anti-AID antibody, quantitative PCR) or indirect methods. The increased level of expression and/or activity refers to level of expression and/or activity in a sample (i.e. sample from the subject) which is at least 10% higher, in an other embodiment at least 15% higher, in an other embodiment at least 20% higher, in an other embodiment at least 25%, in an other embodiment at least 30% higher, in a further embodiment at least 40% higher; in a further embodiment at least 50% higher, in a further embodiment at least 60% higher, in a further embodiment at least 100% higher (i.e. 2-fold), in a further embodiment at least 200% higher (i.e. 3-fold), in a further embodiment at least 300% higher (i.e. 4-fold), relative to the reference expression and/or activity (e.g., in corresponding normal adjacent tissue or alternatively, in a define group of subject).

As used herein, a “substantially similar level” refers to a difference in the level of expression or activity between the level determined in a first sample (e.g., sample from the subject) and the reference expression and/or activity which is less than about 10%; in a further embodiment, 5% or less, in a further embodiment, 2% or less.

As used herein, “aberrant AID expression and/or activity” refers to an increased expression of AID compared to equivalent normal tissue.

As used herein the term “AID-positive tissue” refers to tissue containing cells in which expression and/or activity AID is detectable.

As used herein the term “AID-positive tumor” refers to a tumor containing cells (e.g., cancer cells) in which expression and/or activity AID is detectable.

### Subjects Stratification Methods

The methods of the present invention may also be used for classifying or stratifying a subject into subgroups based on AID expression and/or activity enabling a better characterization of the subject disease and eventually a better selection of treatment depending on the subgroup to which the subject belongs.

In one aspect, the present invention provides a method for stratifying a subject, said method comprising: (a) determining the expression and/or activity of AID in a sample from the subject, (b) comparing said expression and/or activity to a reference expression and/or activity; and (c) stratifying said subject based on said comparison.

The invention provides a method for stratifying a subject based on the expression and/or activity of AID as determined in a tissue sample (e.g., a biopsy) from the subject using the assays/methods described herein.

In another aspect, the present invention provides a method for stratification of a subject having cancer, said method comprising: (a) detecting an expression and/or activity of AID in a sample (e.g., a tumor sample) from the subject, and (b) stratifying said subject based on said detection or absence of detection; wherein the detection (i.e. presence) in said sample is indicative that said subject is suitable for a treatment with an Hsp90 inhibitor of the present invention.

### Combination of Therapies

In an embodiment, the above-mentioned prevention/treatment comprises the use/administration of more than one (i.e. a combination of) therapies (e.g., active/therapeutic agent (e.g., an agent capable of inhibiting AID expression and/or activity)). The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one prophylactic or therapeutic agent in the course of a coordinated treatment to achieve an improved clinical outcome. Such co-administration may also be coextensive, that is, occurring during overlapping periods of time. For example, a first agent may be administered to a subject before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat the disorder in question (e.g., an anticancer agent).

Currently used combined therapies for treating cancer include the administration of radiation therapy with therapeutic antitumoral agents (e.g., imatinib in cancer).

### Hsp90 Inhibitors Combined Treatment in AID Positive Tumors

In one embodiment, the treatment of an AID-positive tumor with a compound reducing the expression and/or activity of AID is combined with at least one other anticancer agent in order to reduce tumor progression and/or development drug resistance.

More specifically, in one embodiment, at least one Hsp90 inhibitor is used in combined chemotherapy for the treatment of AID-positive cancer. In specific aspects of the present invention, an Hsp90 inhibitor (e.g., 17-MG) is combined to at least one of Bay 43-9006, paclitaxel, gemcitabine, cisplatin, docetaxel (Taxol™) (Taxotere™), and AraC for the treatment of AID-positive solid tumors or to imatinib mesylate (Gleevec) for subjects with AID-positive chronic myeloid leukemia (CML) or AID-positive ALL.

In yet other embodiments, at least one Hsp90 inhibitor is used in combination with velcade (bortezomib) for the treatment of relapsed refractory AID-positive multiple myeloma or refractory hematologic AID-positive cancer; or with Herceptin for the treatment of refractory AID-HER2-positive metastatic breast cancer.

### Dosage

The amount of the agent or pharmaceutical composition which is effective in the prevention and/or treatment of a particular disease, disorder or condition (e.g., cancer) will depend on the nature and severity of the disease, the chosen prophylactic/therapeutic regimen (i.e., compound, DNA construct, protein, cells), systemic administration versus localized delivery, the target site of action, the patient's body weight, patient's general health, patient's sex, special diets being followed by the patient, concurrent medications being used (drug interaction), the administration route, time of administration, and other factors that will be recognized and will be ascertainable with routine experimentation by those skilled in the art. The dosage will be adapted by the clinician in accordance with conventional factors such as the extent of the disease and different parameters from the patient. Typically, 0.001 to 1000 mg/kg of body weight/of subject per day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a subject, in the context of the present invention should be sufficient to effect a beneficial prophylactic and/or therapeutic response in the patient over time. The size of the dose will also be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

### Adjustment of Dose of AID Inhibitors

In one embodiment of the present invention, the dose of the at least one Hsp90 inhibitor (e.g., 17-AAG) administered to inhibit AID, is adjusted to the level of AID in the sample (e.g., tumor tissue).

In another aspect, the present invention provides a method for adjusting a treatment, for example the dose of an Hsp90 inhibitor to administer to a subject. Such method comprising: (a) determining the expression and/or activity of AID in a sample from said patient; (b) comparing said expression and/or activity to a reference expression and/or activity of AID determined in a biological sample obtained from said patient at an earlier time (e.g., at the start of treatment); wherein a decrease in said expression and/or activity relative to a corresponding expression and/or activity of AID determined in a biological sample obtained from said patient at an earlier time (at the start of treatment) is indicative that the dose of the at least one Hsp90 inhibitor administered is appropriate whereas a similar level or an increase of AID expression over time is indicative that the dose of the at least one Hsp90 inhibitor administered to the subject should be increased.

### Pharmaceutical Composition

The invention also provides a pharmaceutical composition (medicament) comprising at least one agent of the invention (e.g., an Hsp90 inhibitor), and a pharmaceutically acceptable diluent, carrier, salt or adjuvant. Such carriers include, for example, saline, buffered saline, dextrose, water, glycerol, ethanol, and combinations thereof. The pharmaceutical composition may be adapted for the desired route of administration (e.g., oral, sublingual, nasal, parenteral, intravenous, intramuscular, intraperitoneal, aerosol).

The invention also provides pharmaceutical compositions which comprise one or more agent(s) modulating AID expression and/or activity. Typically, the expression and/or activity of AID is decreased or inhibited. The invention also provides pharmaceutical compositions which comprise one or more agent(s) modulating AID expression and/or activity in combination with at least one other anticancer treatment such as cyclopamine, CUR0199691, Etoposide, Camptothesin, Cisplatin™, Oxaliplatin™ and their derivatives, cyclophosphamide compound (Cy), 13-cis retinoic acid, histone deacetylase inhibitor (SAHA), nucleotide analogues (e.g., 5-fluoro uracyl, azacitidine (Vidaza), Gemcitabine (Gemzar), cytarabine (Ara-C)), kinase inhibitors (e.g., imatinib), etc.

In one embodiment of the present invention, topic treatment (e.g., in nasal mucosa) with at least one Hsp90 inhibitor is provided to alleviate allergies by reducing the AID-dependent switching from IgM to IgE antibody production in B cells.

In one embodiment of the present invention, a treatment with at least one Hsp90 inhibitor is administered in combination with at least one compound having an adverse effect of increasing AID expression and/or activity in cells (e.g., estrogen).

### Kit or Package

The present invention also provides a kit or package comprising the above-mentioned inhibitor or pharmaceutical compositions. Such kit may further comprise, for example, instructions for the prevention and/or treatment of an AID-associated disease (e.g., cancer or autoimmune disease), containers, devices for administering the agent/composition, etc.

The present invention also provides a kit or package comprising a reagent useful for determining AID expression and/or activity (e.g., a ligand that specifically binds AID polypeptide such as an anti-AID antibody, or a ligand that specifically binds a AID nucleic acid such as an oligonucleotide). Such kit may further comprise, for example, instructions for the prognosis and/or diagnosis of cancer, control samples, containers, reagents useful for performing the methods (e.g., buffers, enzymes), etc.

As used herein the term “subject” is meant to refer to any animal, such as a mammal including human, mice, rat, dog, cat, pig, cow, monkey, horse, etc. In a particular embodiment, it refers to a human.

A “subject in need thereof” or a “patient” in the context of the present invention is intended to include any subject that will benefit or that is likely to benefit from the decrease in the expression or activity of AID. In an embodiment, a subject in need thereof is a subject diagnosed as overexpressing AID.

As used herein, the term “a” or “the” means “at least one”.

Although the present invention has been described hereinabove by way of specific embodiments thereof, it can be modified, without departing from the spirit and nature of the subject invention as defined in the appended claims.

The present invention is illustrated in further details by the following non-limiting examples.

In the following examples is described and characterized the constitutive stabilization of AID in the cytoplasm by the Hsp90 pathway of molecular chaperones. Although Hsp90 may also contribute to the biogenesis of AID, it is clear from results presented herein that it largely determines the overall steady state levels of functional AID. The mechanism seems evolutionary conserved since it was active in chicken, mouse and human cells.

### Example 1

### Materials and Methods

### DNA Constructs.

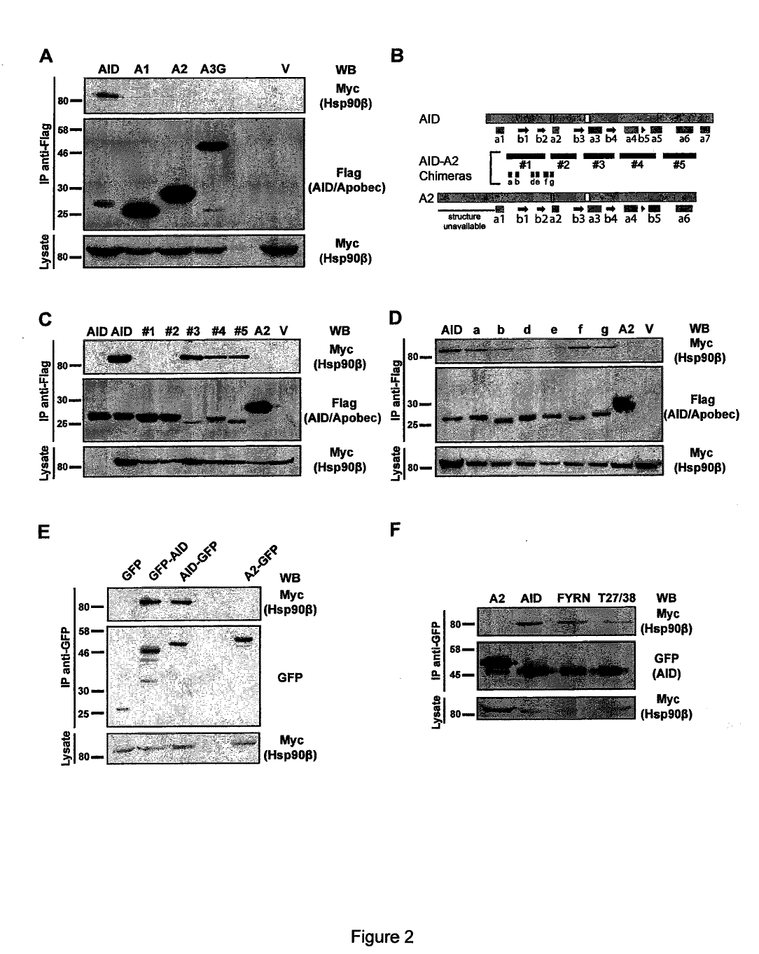

The expression pEGFP-N-3-based (Clontech) vectors for human AID-GFP, AID FYRN-GFP and AID-Flag/HA, as well as for APOBEC2 and AID-APOBEC2 chimeras have been described²⁸. Rat APOBEC1 and human APOBEC3G cloned in pEGFP-C3 as well as human AID T27A/T38A, which was subcloned into pEGFP-N3, were a kind gift of Dr S. Conticello (MRC Laboratory of Molecular Biology, Cambridge, UK)²⁹. To construct N-terminally flag-tagged versions of APOBEC1, APOBEC2 and APOBEC3G, EGFP was excised from pEGFP-C3 using NheI and XhoI and replaced by the annealed oligonucleotides AO1 and AO2. To construct C-terminally flag-tagged versions of some of the proteins, EGFP was excised from pEGFP-N3 using EcoRI and NotI and replaced by the annealed oligonucleotides OJ215 and OJ216. AID under the relatively weak EF1alpha promoter of pEF was subcloned as an NheI-NotI fragment from pEGFP-N3. AID-APOBEC2 chimeras #1 and #2 (described in FIG. 2 B) were excised from pTrc99a²⁸ by partial digestion with NotI and EcoRI and subcloned into the pMXs retroviral vector. Mouse AID (A kind gift from Dr R Harris, U. of Minnesota, MN) was excised from pEGFP-N3 using EcoRI and NotI and subcloned into pMXs. pcDNA3.1 Flag-human Hsp90alpha was inserted as a KpnI and NotI fragment into pcDNA3.1). Myc-human Hsp90beta in pCMV-3Tag2 was a kind gift of Dr J-P Gratton (Institut de recherches cliniques de Montréal (IRCM), Montréal). pcDNA3.1 Myc-human CHIP and HA-ubiquitin were a kind gift of Dr L Petrucelli (Mayo Clinic, Jacksonville, Fla.). Construct names throughout the manuscript indicate the actual order of the fragments in the fusion proteins.

### Reagents

Stock aliquots of 2 mM Geldanamycin, 2 mM 17-MG 5 mM H-89 and 25 mM Forskolin (LC labs, Woburn, Mass.) as well as 50 mM IBMX (Sigma-Aldrich, St Louis, Mo.) in DMSO were stored at −20° C. protected from light. Stocks of 5 mM MG132 (Calbiochem, Gibbstown, N.J.) and 25 microg/mL leptomycin B (LC labs, Woburn, Mass.) in ethanol were stored at −20° C. Cycloheximide (Sigma-Aldrich, St Louis, Mo.) was freshly prepared before each experiment 100 mg/mL in ethanol. Stock of 2 mM Imatinib (Gleevec®, Novartis) in PBS was a kind gift of Dr T Moroy and Dr C Khandanpour (IRCM). All these drugs were stored at −20° C. protected from light.

### Cells and Cell Lines

HeLa cells stably expressing AID-GFP were generated by transfecting pEF-AID-EGFP using TransIT®-2020 Transfection Reagent (Mirus). Puromycine (2.5 microg/mL) was added to the medium 48 h post-transfection. Colonies were picked a week later and puromycine selection was maintained for 2 more weeks. Expression of AID-GFP was verified by flow cytometry and western blot. The Ramos cell lines stably expressing GFP, AID-EGFP and AID-Flag/HA have been described elsewhere²⁸. Ramos cells expressing Myc-CHIP were generated by transfecting with pcDNA3.1 Myc-CHIP and selecting with G418. Positive clones were identified by western blot and subclones from 4 independent myc-CHIP Ramos transfectants obtained by single cell deposition using FACS. Ramos cells stably expressing chimeras AID-A2#1 and #2, DT40 cells stably expressing GFP or AID-GFP as well as the CML cell line K562 (a kind gift of Dr Moroy and Dr Khandanpour, IRCM) stably expressing AID-ires-GFP or GFP control, were obtained by retroviral delivery of these genes cloned in pMXs vectors. The supernatant of HEK293T cells cotransfected at a 3:1:1 ratio with pMX and vectors expressing MLV Gag-Pol and VSV-G envelope, respectively, was used to infect 10⁶ cells in the presence of 8 microg/mL polybrene and 10 mM Hepes. Spin infection was performed at 600 g for 1 h at RT. Infected cells were detected by GFP expression and FACS sorted to obtain homogeneous populations. Primary B-cells from aid−/− mice (a kind gift of Dr T Honjo, U of Kyoto, Japan) were prepared as described^{28,76}. Primary human B-cells were purified from PBMC from voluntary donor blood samples using Ficoll gradient. Resting B-cells were isolated using a B-cell isolation kit from Miltenyi Biotech. B-cells were subsequently activated with recombinant hIL-4 (5 ng/mL; Peprotech) and recombinant human sCD40L (5 microg/mL) as previously described⁷⁷. Work with human samples was according to the guidelines of the ethics committee at the INRS-Armand-Frappier and IRCM (certificate 2009-24).

### Identification of AID Interacting Proteins

5×10⁹ Ramos B cells expressing AID-Flag/HA or empty vector were pelleted, incubated on ice for 10 min and resuspended in Hypotonic Buffer I (Tris 1 mM pH7.3, KCl 10 mM, MgCl₂ 1.5 mM, beta-mercapthoethanol). Cells were centrifuged at 2500 rpm for 10 min at 4° C. and lysed by adding Hypotonic buffer II (Tris 1 mM pH7.3, KCl 10 mM, MgCl₂ 1.5 mM, TSA 1 mM, beta-mercapthoethanol, PMSF 0.5 mM and protease inhibitors (Sigma-Aldrich)). The lysate was centrifuged at 3900 rpm for 15 min at 4° C. and the supernatant recentrifuged at 35000 rpm for 1 h and dialyzed against Tris 20 mM pH7.3, 20% Glycerol, 100 mM KCl, 50 microM beta-mercapthoethanol, 0.5 mM PMSF. The dialyzed lysate was incubated with 150 microL anti-Flag®M2 affinity gel (Sigma-Aldrich) overnight at 4° C. and then extensively washed and eluted using 3× Flag® peptide (Sigma-Aldrich). The eluate was incubated with anti-HA beads (Santa Cruz, San Diego, Calif.) overnight at 4° C. and then washed and eluted using HA peptides (Covance PEP-101P). Protein was concentrated using StrataClean™ Resin (Stratagene) prior to loading on 4-12% gradient precast gel (Invitrogen) for SDS-PAGE. The gel was silver stained, each lane divided into 20 slices and the slices submitted for triptic digestion and peptide identification by mass spectrometry to the IRCM Proteomics service using linear quadrupole IT Orbitrap hybrid mass spectrometer (ThermoFisher). Peak generation and protein identification were done using MASCOT software package.

### Immunoprecipitation and Western Blot.

HEK293T cells cotransfected at a 1:1 ratio with GFP and Myc or Flag-tagged versions of the indicated proteins were homogenized in Lysis Buffer (20 mM Tris pH 8.0, 137 mM NaCl, 10% Glycerol, 2 mM EDTA, 1% TritonX-100, 20 mM NaF) 48 h post-transfection and immunoprecipitations with anti-Flag®M2 affinity gel (Sigma-Aldrich) were performed as described previously (Patenaude et al. 2009). Immunoprecipitation of GFP-tagged proteins were performed using the microMACS™ GFP Isolation kit according to the manufacturer instructions. The eluates and lysates were analyzed by western blot with 1:3000 anti-eGFP-HRP (Miltenyi Biotec), 1:3000 anti-Myc-HRP (Miltenyi Biotec), 1:3000 anti-Flag-HRP (Sigma-Aldrich) or 1:3000 anti-Hsp90 (BD Biosciences) followed by 1:5000 goat anti-mouse-HRP (Dakocytomation). Western blots were developed using SuperSignal™ West Pico Chemiluminiscent substrate (Thermo Scientific). Indicated cells were treated with 10 microM MG132 for 30 min and/or 2 microM GA or DMSO for 5 h before lysis. Human and chicken AID were detected using 1:1000 anti-AID (Cell signaling) followed by 1:5000 goat anti-rat-HRP (Chemicon). Actin was used as loading control by probing with 1:3000 anti-actin (Sigma-Aldrich) followed by 1:10000 anti-rabbit-HRP (Dakocytomation). Endogenous ubiquitin was detected using 1:1000 anti-mono and polyubiquitinylated conjugates antibody (Enzo Life Sciences, Plymouth Meeting, Pa.) followed by 1:5000 goat anti-mouse-HRP (Dakocytomation). Hsp90 isoforms were detected using 1:1000 anti-Hsp90alpha (StressMarq) followed by 1:5000 anti-mouse-HRP (Dakocytomation) or with 1:1000 anti-Hsp90beta (StressMarq) followed by 1:10000 anti-rabbit-HRP (Dakocytomation). CHIP was detected using 1:1000 monoclonal anti-CHIP (Sigma-Aldrich) followed by 1:5000 anti-mouse-HRP (Dakocytomation).

### Monitoring of AID Stability.

In cell lines stably expressing GFP-tagged AID or AID mutants, the GFP fluorescence signal was measured by flow cytometry at various time points after the indicated treatments. Cells were stained with propidium iodide to exclude dead cells from the analysis. For protein synthesis inhibition the cells were incubated in 100 microg/mL cycloheximide for 30 min prior to addition of 2 microM GA or 50 ng/mL LMB. To follow the fate of endogenous AID, 5×10⁶ Ramos or DT40 cells in 5 mL culture medium were treated with GA and 1.5×10⁶ cells aliquots harvested at various time point. Alternatively, 2×10⁶ CH12-F3 cells (a kind gift of Dr T. Honjo, Kyoto University through Dr A Martin, University of Toronto)⁷⁸ were stimulated with 2 ng/mL recombinant human TGFbeta1 (R&D Systems), 20 ng/mL recombinant murine IL-4 (Peprotech) and 5 microg/mL functional grade purified anti-mouse CD40 (Biosciences) for 24 h before GA treatment to initiate the experiment. Cells were washed once with PBS and lysed in SDS-PAGE sample buffer. Lysates were analysed by western blot with 1:1000 anti-AID (Cell signaling) followed by 1:5000 goat anti-rat-HRP (Chemicon) or 1:500 anti-mAID (a kind gift of Dr Alt, Harvard U, Boston, Mass.) followed by 1:10000 goat anti-rabbit-HRP (Dakocytomation) and 1:3000 anti-actin (Sigma-Aldrich) followed by 1:10000 anti-rabbit-HRP (Dakocytomation).

### Somatic Hypermutation (SHM Assays) and Ig Gene Conversion

AID-mediated Ig gene conversion was estimated in DT40cre1 cells by monitoring the frequency of sIgM-gain phenotype, which is mediated by repair of a frameshift in the IgVlambda by gene conversion⁷⁹. DT40 sIgM− cells were purified by FACS sorting and grown for about a week in 24-well plates until confluent before addition of Hsp90 inhibitors. This method was favored over using single cell clones because of the effect of Hsp90 inhibition on cell growth. Cells were grown for 3 weeks in the presence of the inhibitors and the sIgM phenotype measured by flow cytometry as described K. AID-mediated somatic hypermutation was monitored using a sIgM+ DT40 line in which the IgV pseudogenes have been ablated (kind gift of Dr H Arakawa and Dr J-M Buerstedde, IMR, Neuherberg, Germany)⁸¹. Cell populations were sorted and grown as above and the sIgM phenotype analyzed by flow cytometry. The mutation load and pattern was determined by sequencing PCR-amplified Vlambda. AID levels in the populations were quantified by western blot after expansion.

### Class Switch Recombination (CSR Assays)

To analyze class switch recombination, CH12F3-2 cells were preincubated with CFSE (Invitrogen) according to manufacturer instructions before activation with 1 ng/mL TGFbeta1 (R&D Systems), 10 ng/mL recombinant murine IL-4 (Peprotech) and 1 microg/mL functional grade purified anti-mouse CD40 (Biosciences). For chronic Hsp90 inhibition, 17-MG was added 4 h post activation and kept for 3 days. For acute Hsp90 inhibition, 17-AAG was added to the medium for 12 h and then the cells were washed twice with PBS and resuspended in fresh normal medium. sIgA expression was monitored 3 days post-stimulation using PE-conjugated anti-mouse IgA antibody (eBioscience). Alternatively, resting B-cells from AID-deficient mice were purified from total splenic lymphocytes by MACS CD43-depletion (Miltenyi Biotech) as previously described²⁸. Cells were preincubated with CFSE (Invitrogen) and subsequently 10⁶ cells/well were seeded in 24-well plates in the presence of 25 microg/ml LPS (Sigma)+50 ng/ml mouse IL4 (Peprotech). Hsp90 inhibitor was added for 12 h at different times post-activation before extensive washes with PBS and resuspension in culture medium. Isotype switching was analyzed 4 days post-activation by flow cytometry after staining with anti-IgG1-biotin (BD Biosciences) followed by APC-conjugated anti-biotin antibody (Miltenyi Biotech) and propidium iodide. All animal work was approved by the IRCM Committee animal protection.

### Example 2

### Identification of a Specific Aid Interaction Partner

Interaction partners were identified using affinity purification.

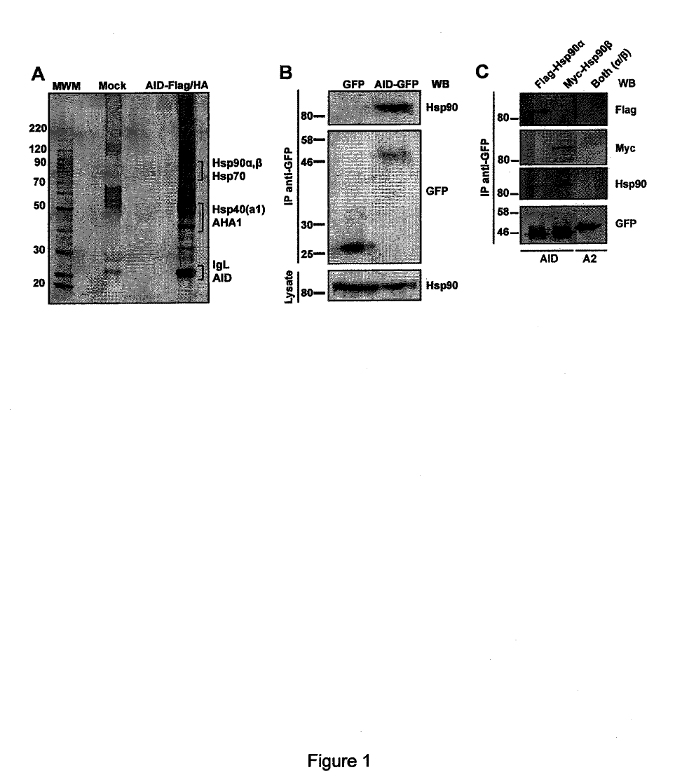

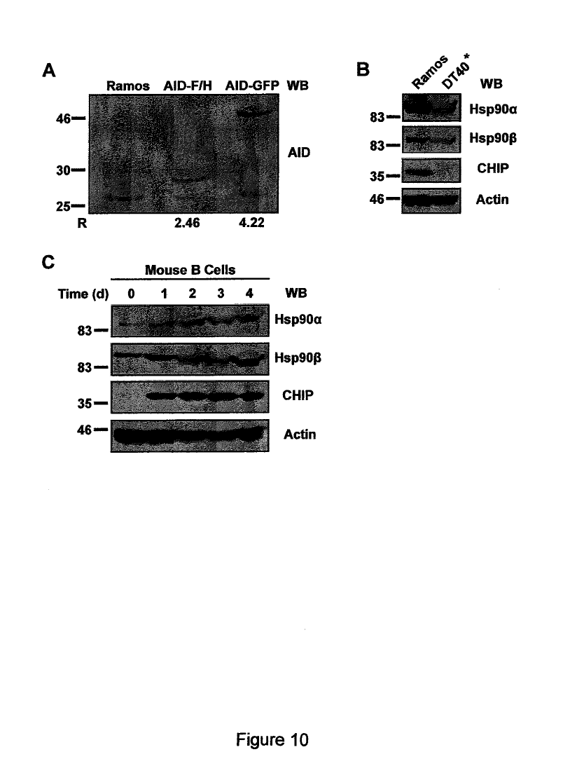

Double immunopurification of AID-Flag/HA from whole cell extracts of stably transfected Ramos B-cells yielded a complex but reproducible pattern of co-purifying proteins (FIG. 1A). Of note, a stable cell line expressing only 2.5-fold of endogenous AID was used (i.e., near physiological conditions and therefore preserving the stoichiometry of protein complexes amount) (FIG. 10). After identification of the pulled-down proteins by mass spectrometry, the presence of several members of the Hsp90 pathway of molecular chaperoning was noticed⁶⁶ including the two cytoplasmic isoforms of Hsp90 (alpha and beta), the Hsp90 cochaperone AHA-1; Hsp70 and one of its Hsp40 chaperones (DnaJa1), as well as several proteasome subunits (see Table III below). All these proteins have been described to exist as a cytosolic complex⁶². Given the importance of Hsp90 in regulating the function and subcellular localization of many signal transduction and shuttling proteins, this interaction was explored further.

The binding of AID to endogenous Hsp90 was confirmed by coimmunoprecipitation of AID-GFP from stably expressing Ramos cells (FIG. 1B). The two major isoforms of Hsp90, alpha and beta are largely redundant but may also have some non-overlapping roles, although this is an active area of research 61,82. Nevertheless, the similar interaction of AID with both Hsp90alpha and beta was confirmed by coimmunoprecipitation (FIG. 1 C). Both isoforms are constitutively expressed in the B-cell lines used as well as in primary mouse B-cells (FIGS. 10 B and C).

The protein levels of Hsp90alpha increased upon cytokine activation in mouse B-cells (FIG. 10 C). These results are in keeping with various reports indicating that growth factors and cytokine signaling, as well as stress, induce Hsp90alpha while Hsp90beta is constitutively expressed^{61,82-84}. Given the high homology between Hsp90 isoforms (˜90% similarity), Hsp90beta was used for interaction studies presented herein but it is expected that AID is a client for both isoforms. The absence of CHIP at day zero indicates that it is induced by cell activation, Day 0 cells not being cycling, but arrested in G1.

TABLE III
Proteins copurifying with AID-Flag-HA
identified by mass spectrometry

| | | Mascot | Coverage | |
|---|---|---|---|---|
| HUGO name | Peptides (n) | Score^{a} | (%) | Description |
| HSP90AB1 | 87 | 2178 | 44 | Heat shock 90 kDa |
| | | | | protein 1, beta |
| HSP90AB1* | | 300 | 8 | |
| HSP90AA1 | 66 | 1668 | 35 | Heat shock 90 kDa |
| | | | | protein 1, alpha |
| HSP90AA1* | | 151 | 6 | |
| HSP90AB2P | 17 | 438 | 16 | Heat shock protein |
| | | | | 90Bb |
| HSP90AB4P | 9 | 202 | 9 | Putative heat shock |
| | | | | protein HSP 90-beta 4 |
| HSPA8 | 47 | 1338 | 39 | Heat shock 70 kDa |
| | | | | protein 8 isoform 1 |
| HSPA6 | 10 | 327 | 8 | Heat shock 70 kDa |
| | | | | protein B′ |
| AHSA1 | 2 | 81 | 3 | AHA1, Activator of |
| | | | | heat shock 90 kDa |
| AHSA1* | 2 | 27 | 9 | protein ATPase |
| | | | | homolog 1 |
| DNAJA1 | 6 | 212 | 26 | Hsp40 homolog, |
| | | | | subfamily A, |
| | | | | member 1 |
| PSMD2 | 9 | 242 | 14 | Proteasome 26S |
| | | | | non-ATPase subunit 2 |
| PSMD1 | 3 | 105 | 2 | Proteasome 26S |
| | | | | non-ATPase subunit 1 |
| PSMD6 | 2 | 105 | 5 | Proteasome 26S |
| | | | | non-ATPase subunit 6 |
| PSMD6* | 2 | 46 | 6 | |
| PSMC2 | 2 | 75 | 3 | Proteasome 26S |
| | | | | ATPase subunit 2 |

*Proteins identified from two independent experiments
^{a}A threshold Mascot score of 35 was defined as cut-off, indicating a 95% confidence of being a true identification.
In the case of AHSA1* the MS profile was examined by hand to confirm the reliability of the observation.

To test the specificity of the interaction between AID and Hsp90, the AID paralog proteins APOBEC1 (identified as A1 in FIG. 2A), APOBEC2 (identified as A2 in FIG. 2A) and APOBEC3G (identified as A3G in FIG. 2A) were used as controls, since they share ˜50-60% similarity with AID⁸⁵. Unlike AID, none of them coimmunoprecipitated Hsp90beta (FIG. 2 A). As a further measure of specificity, the region of AID interacting with Hsp90beta could be mapped to the N-terminal half of the molecule by using AID-APOBEC2 chimeric proteins (FIG. 2 B and C). The interaction of AID with Hsp90beta could be reduced to various degrees, but not abrogated, by smaller replacements of 3-5 amino acids located between position 19-46 of AID (chimeras a to g) with the homologous APOBEC2 positions (FIG. 2 D), nor could it be abrogated by bulky N-terminal fusions like in GFP-AID (FIG. 1E). The region of AID interacting with Hsp90 is also suggested to mediate AID dimerization^{28,30} so it was not unexpected that an AID mutant showing impaired oligomerization²⁸ still interacted well with Hsp90 (FIG. 2 F). Phosphorylation can modulate the binding of Hsp90 to its clients⁸⁶ but both known Protein Kinase A phosphorylation sites within the N-terminal region of AID, Thr27 and Ser38, were dispensable for the interaction (FIG. 2 F). The results suggest that AID specifically binds to Hsp90 through the N-terminal region in an oligomerization and phosphorylation-independent fashion.

### Example 3

### Sensitivity of Aid to Hsp90 Inhibitors

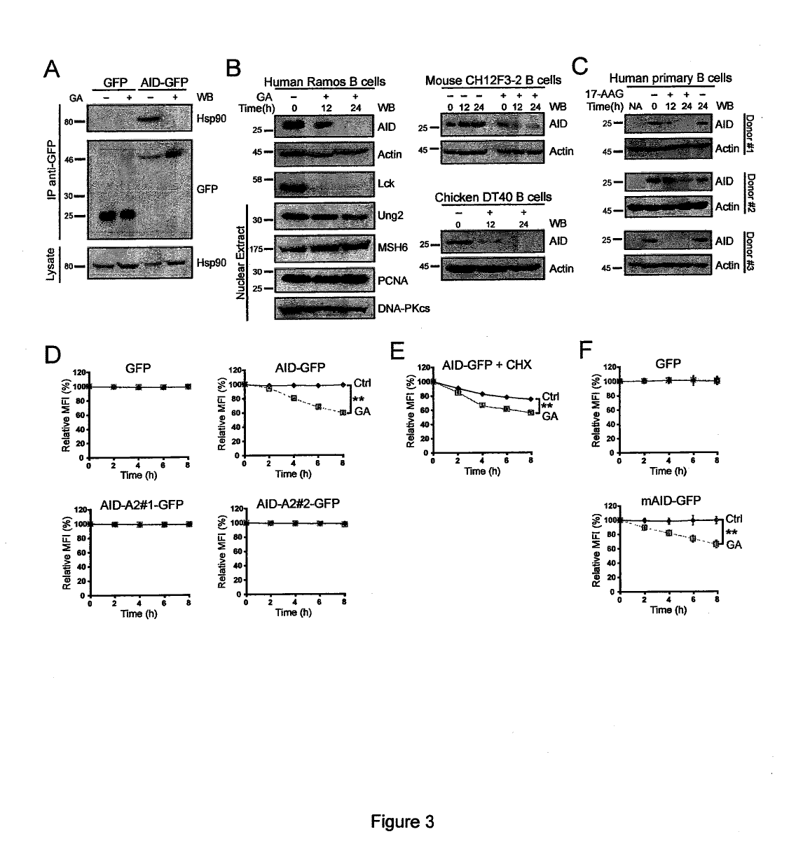

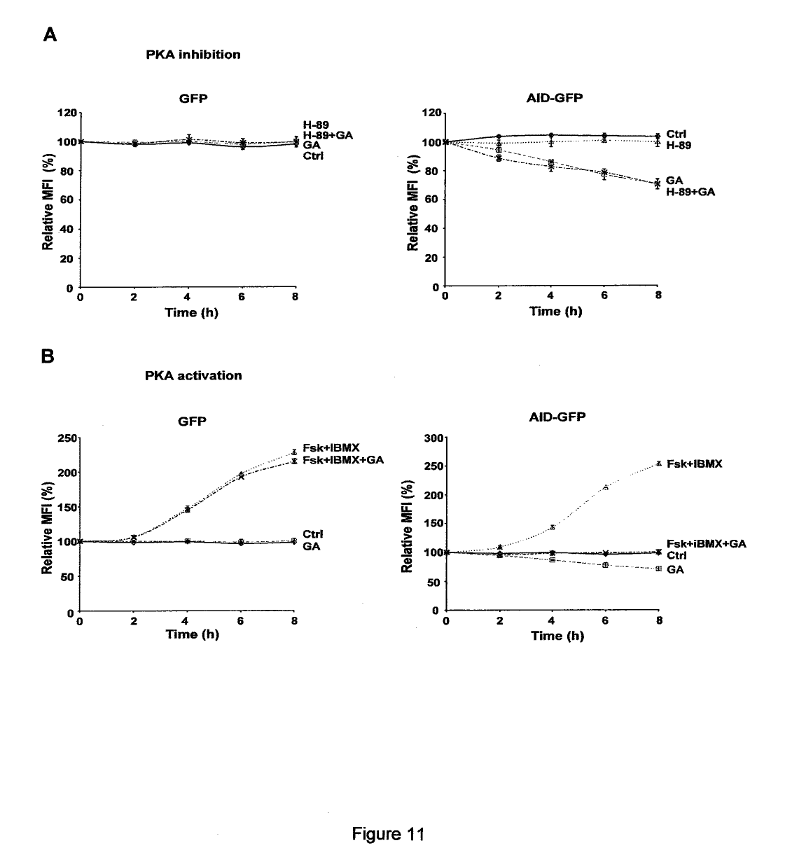

The chaperone activity of Hsp90 relies on an ATP hydrolysis cycle, which can be inhibited by the drugs geldanamycin (GA) and its derivative 17 (Allylamino) geldanamycin (17-MG)^{87,88}. Ramos cells with GA prevented the interaction of AID-GFP with Hsp90 by coimmunoprecipitation (FIG. 3 A). Furthermore, chronic treatment of human, chicken and mouse B-cell lymphoma lines with GA caused a clear reduction in the levels of endogenous AID at 12 and 24 h (FIG. 3 B). The known Hsp90 client kinase Lck was used as a positive control and was also reduced in these conditions. Other enzymes involved in antibody diversification, including UNG2 (uracil-DNA N-Glycolsylase) and MSH6, were not sensitive to Hsp90 inhibition (FIG. 3 B). Endogenous AID in stimulated human primary B-cells from multiple donors was also sensitive to Hsp90 inhibition with the GA derivative 17-AAG, indicating that endogenous AID in non-transformed cells is also stabilized by Hsp90 (FIG. 3 C)). In order to use a more sensitive and quantifiable assay to monitor the decay of AID at shorter times and to be able to compare AID variants, stable Ramos transfectants expressing various AID-GFP constructs were established. These experiments confirmed that AID-GFP, but not GFP, was destabilized by Hsp90 inhibition in these cell lines (FIG. 3 D, 1^{st} and 2^{nd} panels). Treatments inhibiting or exacerbating Protein Kinase A (PKA) activity had no effect on the sensitivity of AID-GFP to GA, further suggesting that these two pathways are not connected (FIG. 11). PKA Phosphorylates AID in two positions that are within the region that binds Hsp90. Also as it would be expected, the AID-A2 chimeras that did not interact with Hsp90 were insensitive to GA treatment (FIG. 3 D, 3^{rd} and 4^{th} panels). The decay kinetics of AID-GFP after pretreating the cells with cycloheximide (CHX) so as to follow the pool of AID that had already been synthesized and not the nascent AID that might be more sensitive to folding requirement was measured (FIG. 3 E). CHX caused the expected decay of AID-GFP which was accelerated by GA, indicating a role for Hsp90 in stabilizing fully synthesized AID. Mouse and human AID-GFP were sensitive to Hsp90 inhibition when retrovirally delivered into mouse splenic B-cells (FIG. 3 F and not shown). Functional Hsp90 appears necessary to maintain the steady state levels of AID in vivo in normal and transformed cells.

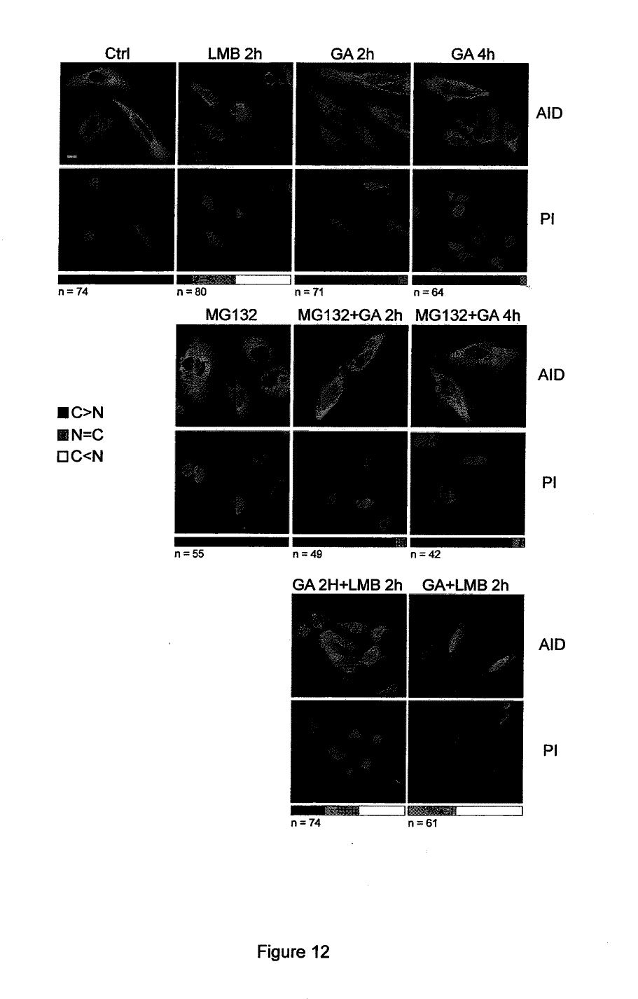

Binding and release from Hsp90 can regulate sub-cellular localization^{89,90}. However, no change in AID localization upon inhibition of Hsp90 was observed indicating that Hsp90 is not the major protein retaining AID in the cytoplasm (FIG. 12 top panels). Simultaneous inhibition of Hsp90 and nuclear export may have a small effect on the speed with which AID accumulates in the nucleus (FIG. 12 bottom panels). Hsp90 could therefore have a minor contribution in retaining a fraction of AID in the cytoplasm. Alternatively, a proportion of the Hsp90-bound AID might be posed to adopt a functional conformation. Then, synchronized release of AID from Hsp90 by GA treatment would lead to an apparent increase in nuclear import of uncertain functional relevance.

The effect of treating Ramos B-cells expressing AID-GFP with GA in combination with leptomycin B (LMB) was examined. LMB causes AID-GFP to accumulate in the nucleus^{58,59} where it is destabilized⁶⁰. LMB is a non-specific inhibitor of nuclear export. When AID is translocated into the nucleus, it is either actively destabilized or just less stable because they are not protected by cytoplasmic factors such as Hsp90. LMB is irreversible and cytotoxic. Although the effect of LMB on endogenous AID and the LMB dose response for AID are currently unknown, an increase of AID in the nucleus is expected to cause an increase in AID derived mutations even if it is destabilized^{58,91}.

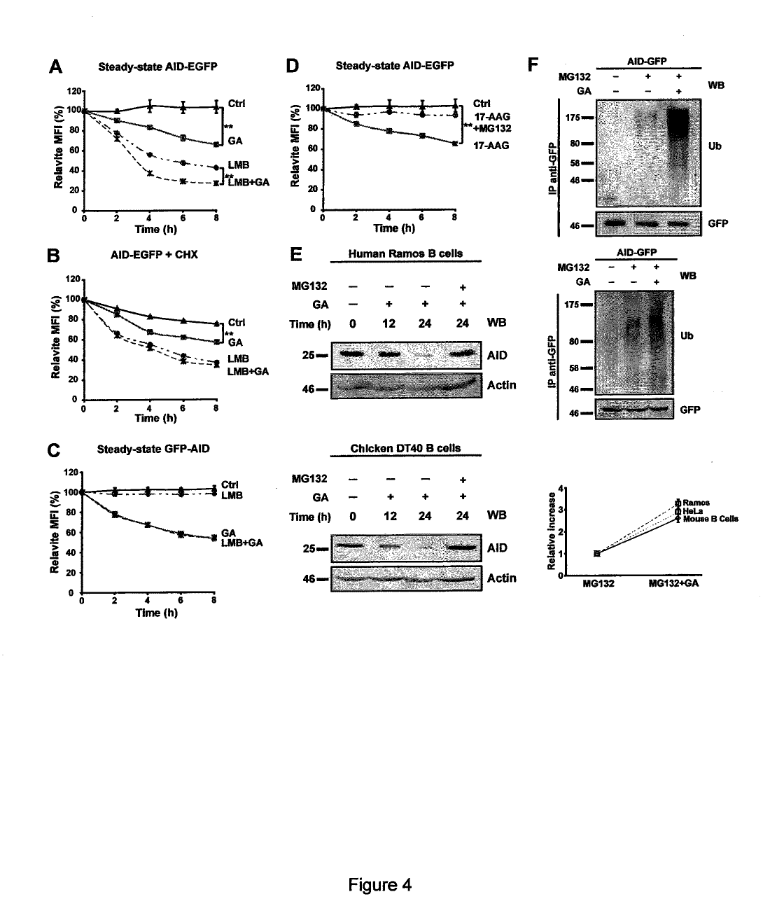

The kinetics of AID-GFP decay following GA or LMB treatment were different, with GA showing a less rapid effect than LMB and the effects being additive when both drugs were combined (FIG. 4 A).

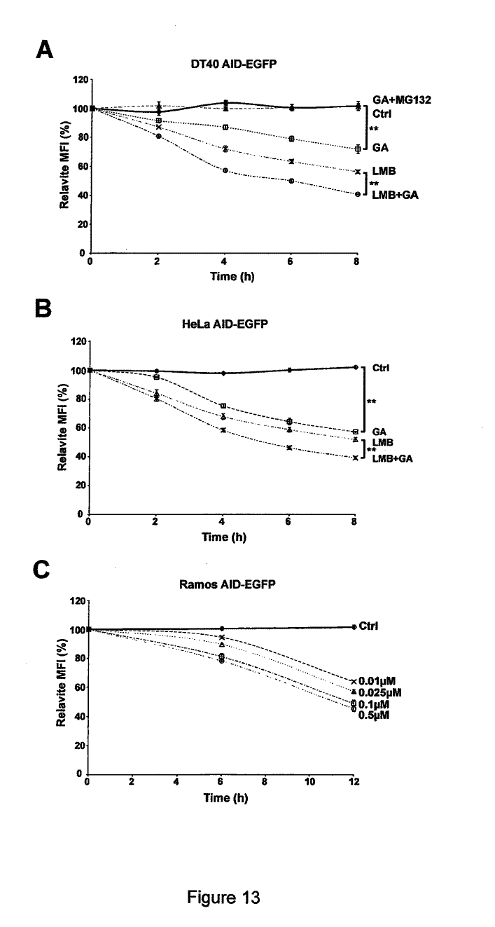

Similar experiments were performed after pre-treating the cells with cycloheximide (CHX) so as to follow the pool of AID that had already been synthesized, and not the nascent AID that might be more sensitive to folding requirements. Again, both GA and LMB treatments resulted in different AID-decay kinetics but, interestingly, the combined treatment was not different from that when LMB was used alone i.e. nuclear export inhibition has the maximum effect on its own and further Hsp90 inhibition does not cause any further decrease (FIG. 4 B). These treatments seem to distinguish two fractions of cytoplasmic AID. Importantly, these experiments also show that Hsp90 not only participates in folding AID, but is important for stabilizing the existing AID pool since GA has an effect on its own even on the CHX treated cells (where there is no newly synthesized AID). The lack of detectable nuclear translocation of AID after Hsp90 inhibition, together with the different kinetics and additive effects of GA and LMB, suggests that each treatment destabilizes AID by a different pathway. Identical results were obtained using DT40 and Hela cells stably expressing AID-GFP (FIGS. 13 A and B).

As demonstrated (i.e., FIG. 4D) in different hematopoietic- and non-hematopoietic-derived cell lines, AID protein is sensitive to treatment with GA and 17 AAG, well-known Hsp90 inhibitors. The data obtained shown that functional Hsp90 is necessary to maintain the steady state levels of AID in vivo in normal and transformed cells.

### Example 4

### Treatment with Hsp90 Inhibitors Decrease the Level of Aid in the Cytoplasm

The present assay sought to determine whether the different responses in AID decay observed after Hsp90 or nuclear export inhibition reflected different compartmentalization of the destabilization pathways.

Ramos cells expressing GFP-AID were used to demonstrate that Hsp90 stabilizes cytoplasmic AID. The N-terminal GFP fusion (GFP-AID) completely blocks nuclear import of AID²⁸ but not its binding to Hsp90 (FIG. 2 E). GFP-AID was not destabilized by treatment with LMB (an indirect AID inhibitor that leads to AID degradation by sending AID to the nucleus where is less stable than in the cytoplasm) (FIG. 4 C) but it was still sensitive to GA treatment. Hsp90 clients are usually degraded through the proteasome^{63,92}. Indeed, the proteasome inhibitor MG132 prevented the degradation of AID induced by Hsp90 inhibition; both for AID-GFP in stable transfectants of Ramos and DT40 cells (FIG. 4 D and 13), as well as for endogenous AID in the same cell types (FIG. 4 E). Identical results were obtained with a second proteasome inhibitor, lactacystin (not shown). Treatments leading to proteasomal degradation of AID caused also its polyubiquinylation. A reproducible ˜3.5-fold increase in AID polyubiquitinylation was observed after combined inhibition of the proteasome and Hsp90 versus inhibiting only the proteasome in Ramos and primary mouse B-cells (FIG. 4 F). This pathway was not particular to B cells since it was also observed for AID-GFP in stably transfected HeLa cells (FIG. 4 F and not shown).

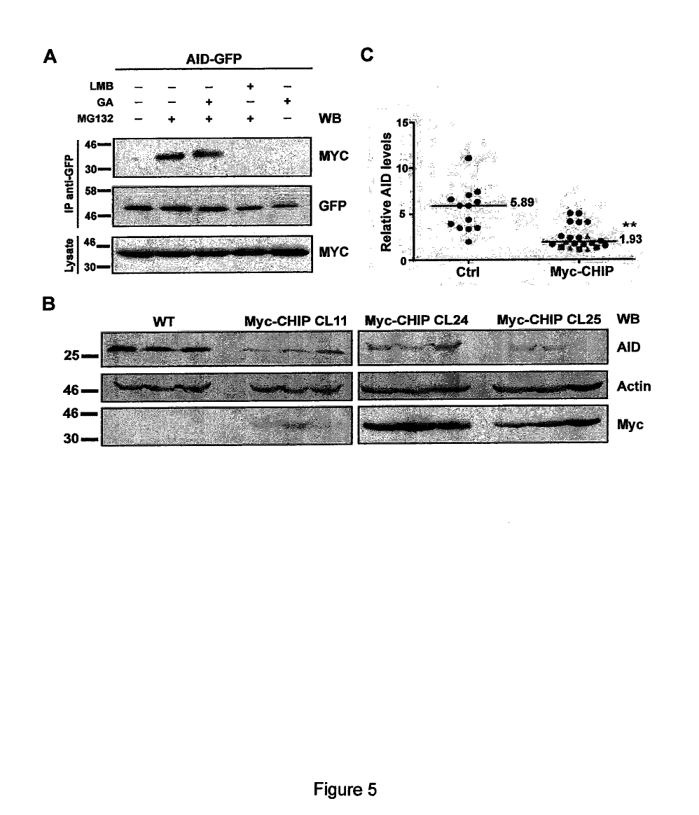

The E3-ubiquitin ligase CHIP is associated with Hsp90 and mark many Hsp90 clients for degradation⁹³. The following assay sought to determine whether AID could be a substrate for CHIP. Interaction of AID with CHIP could be demonstrated by coimmunoprecipitation from cell extracts of HeLa stably expressing AID-GFP (FIG. 5 A). The interaction was only apparent when the cells were pretreated to inhibit the proteasome, which allows the accumulation of this high turn over interaction⁹⁴. Of note, CHIP is expressed in Burkitt's lymphoma cell lines and induced upon activation in primary B-cells (FIG. 10). This assay sought to determine whether the overexpression of CHIP would lead to overall decreased levels of AID, by changing the balance of the equilibrium between stabilization and degradation of this pathway. Indeed, several independent transfectants of Ramos B-cells expressing myc-CHIP showed a significantly reduced steady state level of AID (FIG. 5 B and C). This is further proof that the Hsp90 pathway stabilizes AID.

Altogether, these results indicate that cytoplasmic AID requires constant maintenance by the Hsp90 chaperone and that altering the balance of this reaction, either by inhibiting Hsp90 or exacerbating the pathway that leads to degradation through CHIP overexpression, leads to greatly diminished AID levels.

### Example 5

### Treatment with Hsp90 Inhibitors Decreases Aid Shm Activity

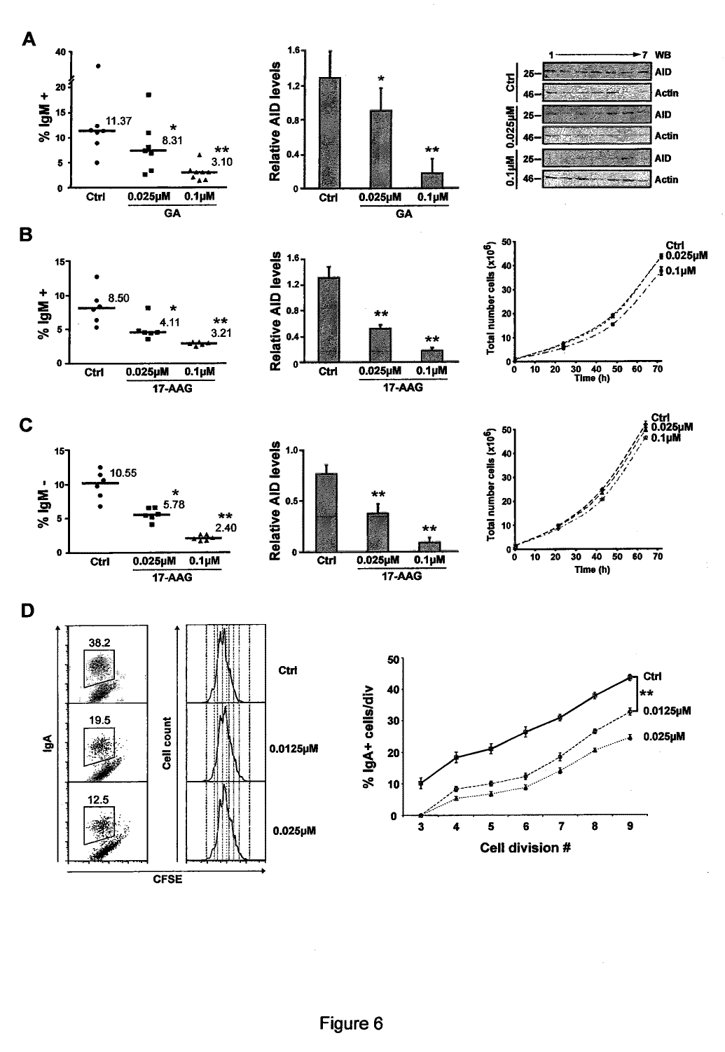

Hsp90 is an essential protein in eukaryotic cells^{95,96}, which precludes its genetic ablation or complete inhibition for the relatively long periods of cell culture required to test antibody gene diversification. Two strategies were used to overcome this. First, for IgVlambda (Ig variable region) diversification assays, which take several weeks, a chronic treatment with low doses of Hsp90 inhibitors, compatible with sustained cell growth was used. Since the decay of AID caused by GA was dose dependent (FIG. 13 C), this assay sought to determine whether suboptimal inhibition of Hsp90 would still lead to a proportional decrease in AID levels, which could still impact on the efficiency of antibody diversification. The effect of Hsp90 inhibition on IgVlambda diversification was first tested using DT40 cells, a chicken B cell line that diversifies the variable region of its antibody genes by Ig gene conversion i.e. an AID-dependent mechanism that is initiated just as SHM but is resolved by homologous recombination-like repair by copying fragments of similar genes located upstream from the IgV region. A dose dependent reduction of IgVlambda gene conversion was observed in GA-treated DT40 cells, monitored by fluctuation analysis of sIgM expression; which was proportional to the reduction in AID levels (FIG. 6 A). However, GA still caused delayed cell growth (cytotoxicity), even at these low doses (not shown). Similar experiments were then performed using the less toxic 17-AAG⁹⁷, which at low doses had minimal impact on cell growth while still causing a robust decrease in AID levels and a proportional inhibition of IgVlambda gene conversion (FIG. 6 B).

Analogous results were obtained using another DT40 cell line that has been engineered to ablate the upstream donor genes and is therefore unable to produce Ig gene conversion, but diversifies the IgVlambda by SHM⁸¹ (FIG. 6 C). The decrease in SHM was confirmed by direct sequencing of the IgVlambda region. This region was PCR-amplified from control and 17-MG-treated (0.1 microM) cell populations after 4 weeks of growth, the PCR product cloned and 10-11 clones for each population were sequenced. The mutation frequency was diminished ˜5-fold in the treated cells compared to the controls (1.11×10⁻³ versus 5.42×10⁻³ mutations/base pair)

These data showed that Hsp90 inhibition by GA or 17-MG treatment decreases in a dose dependent manner the levels of AID and that this leads to a proportional reduction in both mechanisms known to diversify antibody variable region (i.e. IgVlambda gene conversion and SHM). Moreover, these data demonstrated that chronic treatment with a low dose of Hsp90 inhibitor that is compatible with sustained cell growth is able to decrease AID-driven antibody diversification.

### Example 6

### Treatment with Hsp90 Inhibitors Decreases Aid Class Switch

### Recombination Activity

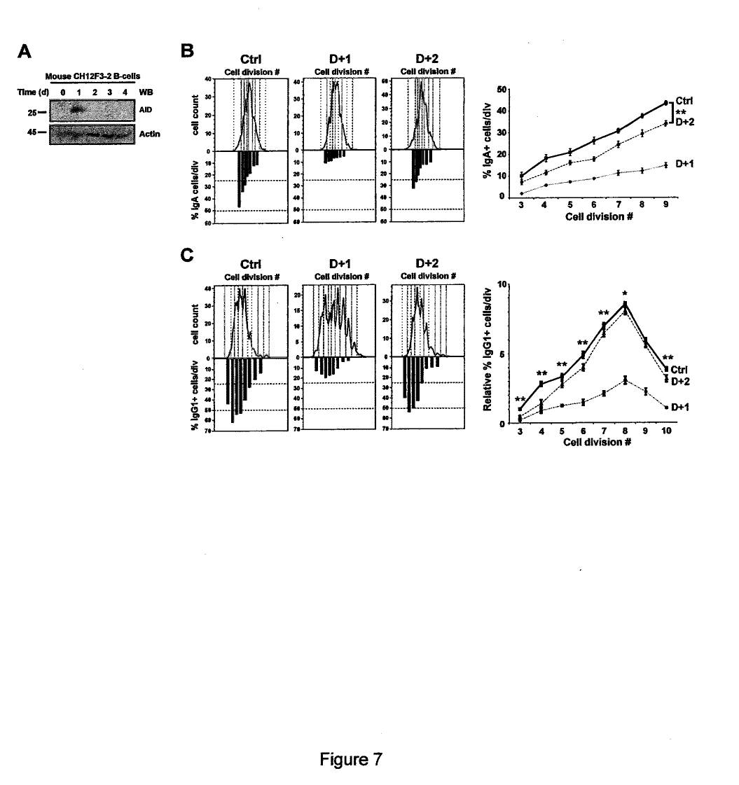

The effect of 17-MG on AID-induced CSR was tested using the mouse CH12-F3 cell line, a B-lymphoma cell line, which efficiently switches from IgM to IgA after cytokine stimulation⁷⁸. To factor in any effect on cell growth, CFSE staining was used to monitor cell proliferation. Since both CSR and AID expression have been shown to be division-linked processes “,”, this allows to compare the efficiency of switching between cells that have undergone the same number of cell divisions, even if Hsp90 inhibition impacts the growth of the cell population. There was a clear and dose-dependent reduction in CSR caused by 17-MG, overall and for each cell division tested (FIG. 6 D). Since AID is induced only transiently after stimulating CH12-F3 cells (FIG. 7 A), a second strategy was used for inhibiting Hsp90, consisting in an acute 12 h treatment with higher doses of 17-MG, after which the drug was removed. A drastic reduction in CSR to IgA was observed when the 17-MG treatment was performed at day 1, when the peak of AID protein is observed (FIG. 7 B). As it would be expected, treating at day 2 had a statistically significant but much milder effect on CSR, compatible with the effect of 17-MG being on AID rather than other factor required for CSR. Essentially the same results were obtained in normal mouse splenic B-cells (FIG. 7 C). Endogenous AID was not detected in mouse splenocytes with the antibodies that were tested. Nevertheless, regardless of AID induction kinetics during the four days of the assay, the detection of surface IgG1 at day 4 should be the consequence of AID expressed early on. In keeping with this, a drastic decrease of CSR to IgG1 was observed for all cell divisions in cells that were treated with 17-MG at day 1 post-stimulation. Again, a smaller but still statistically significant effect was apparent when the cells were treated at day 2 post-stimulation (FIG. 7C). As it would be expected, treating the cells with 17-MG at day three had not effect on the efficiency of CSR observed at day 4 (data not shown).

17-MG treatment decreases in a dose dependent manner the levels of AID-driven class switch recombination activity (e.g., inhibition of IgM to IgA switch and to IgG1).

Implications of the above results on the regulation of AID activity through the regulation of its steady state levels are of relevance. This is particularly relevant because the dose effects of AID on the efficiency of antibody diversification, chromosomal translocations and lymphomagenesis are well documented¹⁰⁻¹⁴. By modulating the half-life of the bulk of AID, Hsp90 determines the availability of functional AID since inhibiting Hsp90 leads to a decrease in antibody diversification that is proportional to the decrease in AID protein (FIG. 6).

### Example 7

### Treatment of Cell with an Hsp90 Inhibitor Reduces Oncogenic Mutations by Aid

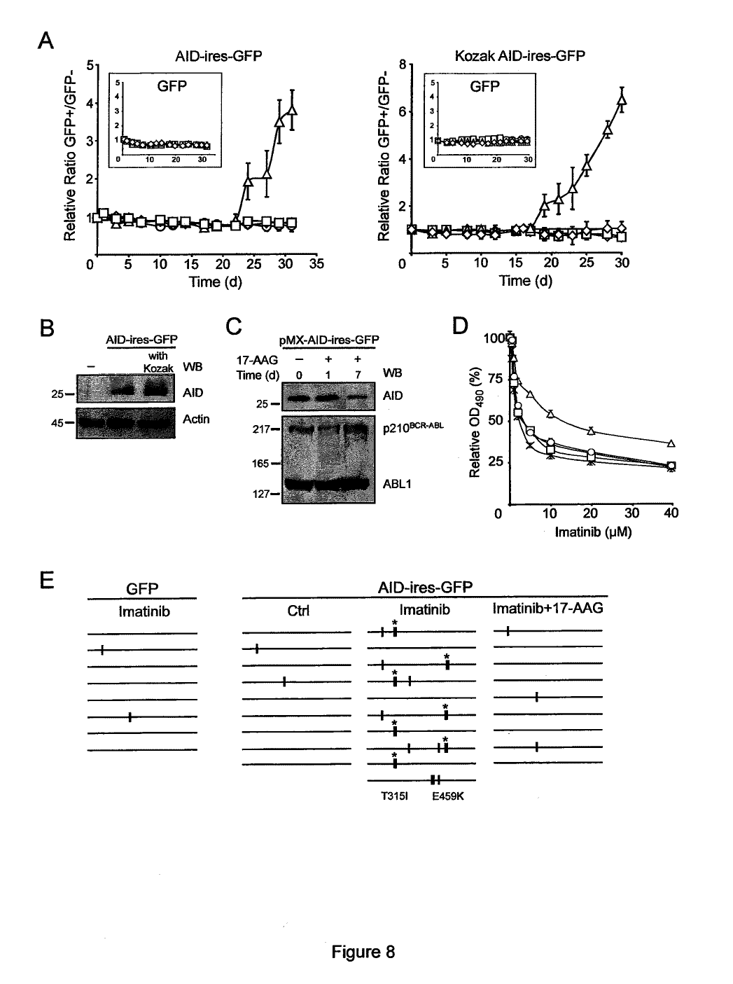

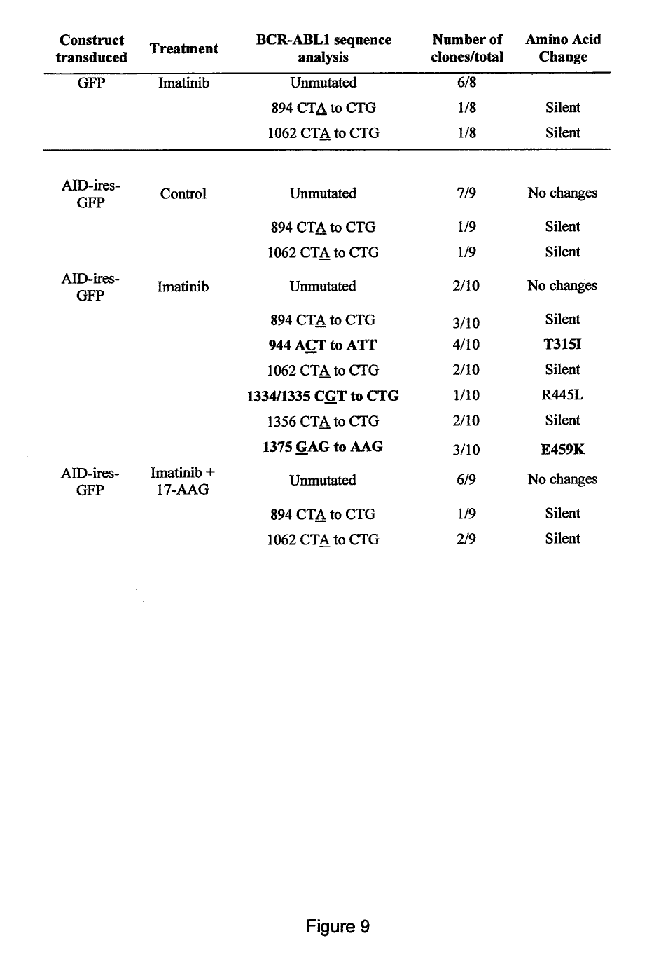

It was recently demonstrated that AID mutates the BCR-ABL1 oncogene in chronic myeloid leukemia (CML) cells, thereby rendering the ABL1 kinase resistant to the current therapeutic drug imatinib²³. The present assay sought to determine whether decreasing the levels of AID by means of chronic Hsp90 inhibition could prevent off-target mutagenesis. For this, the CML cell line K562 was transfected with retroviruses encoding AID-IRES-GFP or control IRES-GFP. Mixed populations of non-transduced cells (GFP−) and transduced cells (GFP+) at 50:50 ratio were prepared for each construct. As it has been shown²³, these populations maintained this ratio during growth unless they were put under selective pressure by adding imatinib to the cultures. Since BCR-ABL1 confers growth advantage, imatinib treatment of AID-expressing cells results in the selection of cells harboring mutated BCR-ABL1 that became resistant to the drug. This translated into a predominance of GFP+ cells in the mixed culture that became apparent during the third week of cell growth (FIG. 8 A, triangles). The K562 cell cultures containing cells expressing AID-GFP turned from a 50:50 to an ˜80:20 ratio of GFP+:GFP− cells by 4 weeks, while the ratio in those cultures expressing only GFP was unaffected (FIG. 8 A inset). This was AID-dependent as the imatinib-resistant cells appeared earlier in cultures expressing higher levels of AID (compare FIGS. 8 A, right and left panel and B). As indicated earlier, the presence of a Kozak sequence (FIG. 8 A, right panel) leads to higher AID expression in transduced CML K562 cells. More importantly, the increase in imatinib resistance, and therefore any effect on the GFP+:GFP− ratio in cultures expressing AID-GFP, was completely prevented by treating the cultures with very low doses of 17-AAG (FIG. 8 A, left and right panels circles). Treatment with the same dose of 17-MG reduced AID but not BCR-ABL1 protein levels in K562 cells transduced with retroviral construct expressing AID-ires-GFP (FIG. 8 C). AID-mediated increase in imatinib IC₅₀ was confirmed; 17-AAG prevented this increase (FIG. 8 D, compare triangles and circles). Mutations in BCR-ABL1 kinase domain was verified by sequencing a 700 bp-fragment in ABL1 kinase domain of BCR-ABL1 (exon13 of BCR and exon 9 of ABL1). An increase in point mutations could be detected in AID-expressing K562 cells growing in imatinib (FIGS. 8 E and 9). In contrast, the mutation level in AID-expressing cultures that were jointly treated with imatinib and 17-MG was indistinguishable from the GFP control (FIGS. 8 E and 9).

The experiment described above shows that low doses of Hsp90 inhibitor (e.g., 17-MG or GA) can prevent (or at least significantly delay) mutations of BCR-ABL1 by AID in CML cells and thereby imatinib resistance. This could have practical implications in the treatment of CML, in which AID is expressed in late stages underpinning drug resistance²³. An analogous role for AID could be hypothesized in those lymphomas in which AID may accelerate progression, such as conversion of follicular lymphoma (FL) or B-CLL into DLBCL⁶⁹ or AIDS-associated B-cell lymphomas⁵⁴. Monitoring of AID levels by a sensitive technique could allow timely combined therapy with Hsp90 inhibitors to delay disease progression.

### Example 8

### Stratification and Follow Up of Patients Having an Aid-Positive Tumor: an Hsp90 Inhibitor Treatment

The measurement of AID expression and/or activity in association with a tumor will be used for patient stratification and follow up. For example, bone marrow and peripheral blood biological samples will be obtained from patients having a chronic myeloid leukemia (CML). The expression of AID in these samples will be measured and compared to those in blood samples of patients that do not have this disease (e.g., healthy patients or patients having diseases other than CML or patients having a different CML subtype). In one control cell population, normal naive B cells (CD19+CD27+IgD+) will be sorted from peripheral blood of healthy donors by flow cytometry using a FACS Vantage™ SE cell sorter (BD Biosciences).

The determination of AID mRNA expression level will then be performed, according to standard conditions, by quantitative real-time PCR carried out with the SYBR™ Green ER mix from Invitrogen (Carlsbad, Calif.) using primers specific for AID mRNAs. During the PCR amplification, the SYBR™ Green ER dye in the mix binds to accumulating double-stranded DNA and generates a fluorescence signal proportional to the DNA concentration that can be visualized and measured using a AB17900HT (Applied Biosystems, Foster City, Calif.) real-time PCR system. The level of AID PCR product measured in the patient sample will be compared to the mean level obtained in the control. A higher level of AID PCR product in the patient sample (e.g., a 10% or a 15% increase or more) will be indicative that the administration of an Hsp90 inhibitor to reduce AID expression and/or activity (e.g., 17-AAG) is appropriate whereas a similar or a lower level will be indicative that the administration of an Hsp90 inhibitor is unnecessary. The administration of an Hsp90 inhibitor to reduce AID expression and/or activity in the patient could be combined to at least one other anti-cancer treatments (e.g., imatinib).

The determination of AID protein expression could also be performed by detecting AID using specific monoclonal/polyclonal antibodies (see Table I above for examples of antibodies) by western blot or other immunological assays including immunocytochemistry, flow cytometry of permeabilized cells, ELISA, etc.

At regular intervals following and during the administration of the Hsp90 inhibitor, patients will be monitored for AID protein expression as described above. The measurement of a stable or higher level of AID expression in the patient sample compared to a control time point sample from the same patient before starting the Hsp90 inhibition treatment will be indicative that a higher dose of Hsp90 inhibitor should be used whereas a lower level of AID protein will be indicative that the dose of Hsp90 inhibitor administered is appropriate and should be maintained.

### Example 9

### Stratification and Follow Up of Patients Having AID Highly Expressed in a B Cell Population: an Hsp90 Inhibitor Treatment

The measurement of AID expression and/or activity in a B cell population of a subject affected with an AID-associated disease (e.g., neoplastic or autoimmune diseases) or in a subject that is likely to develop an AID-associated disease will be used for patient stratification.

In one example, a B cell population sample will be obtained from patients having preneoplastic alterations (e.g., lymphocytosis, lymph node hyperplasia, mutations in oncogenes or in tumor suppressor genes, etc.) or presenting an indolent or non-aggressive form of lymphoma or leukemia.

The expression of AID in these samples will be measured as described in Example 8 above and compared to those of a control sample (e.g., from patients that do not have this disease and/or are not likely to develop the disease). As a control cell population, normal naive B cells (e.g., CD19+CD27+IgD+) will be sorted from peripheral blood of healthy donors by flow cytometry using a FACS Vantage™ SE cell sorter (BD Biosciences). The levels of AID PCR product measured in the patient sample will be compared to the mean level obtained in the control. A higher level of AID PCR product in the patient sample (e.g., a 5%, a 10% or a 15% increase or more) will be indicative that the administration of an Hsp90 inhibitor (e.g., 17-AAG) is appropriate whereas a similar or a lower level will be indicative that the administration of an Hsp90 inhibitor to reduce AID expression and/or activity is unnecessary.

At regular intervals following and during the administration of the Hsp90 inhibitor, patients will be monitored for AID protein expression as described above. The measurement of a stable or higher level of AID expression in the patient sample compared to a control time point sample from the same patient before starting the Hsp90 inhibition treatment will be indicative that a higher dose of Hsp90 inhibitor should be used whereas a lower level of AID protein will be indicative that the dose of Hsp90 inhibitor administered is appropriate and should be maintained.

### Example 10

### Stratification and Follow Up of Patients Having AID Normally Expressed in a B Cell Population but in Combination with Other Predisposing Factors: an Hsp90 Inhibitor Treatment

Stratification also involves the measurement of expression and/or activity of genes known to regulate the AID mutator activity in a B cell (e.g., p53, ATM, Nbs1, UNG, SMUG1, MSH2, MSH6).

Genetic loss-of-function mutations are DNA modifications (e.g., deletions, missense substitutions) leading to a decrease in expression and/or activity of a specific gene. Analysis of DNA for the detection of a loss-of-function mutation in genes known to regulate the AID mutator activity will be performed. Genomic DNA from the relevant B cell population and/or B cell malignancy and/or B cell premalignancy will be obtained from a subject using Gentra Puregen™ Kit (QIAGEN). The exons of the genes under scrutiny (e.g., p53 and ATM) will be amplified by PCR and sequenced to determine the presence of a loss of function mutation by the analysis of the deduced protein (e.g., the introduction of stop codons) as compared to the wild type sequence. Wild type sequences are available in public database but could also be obtained from DNA purified from normal samples. Databases with collection of loss-of-function mutations are also available. For instance, both somatic and germline p53 mutations are compiled in a worldwide database at the International Agency for Research on Cancer¹⁰⁰. Most mutations result in missense substitutions that are scattered throughout the gene but are particularly dense in exons 5-8, encoding the DNA binding domain. In sporadic cancer, environmental or lifestyle exposures (e.g., ultraviolet (UV), tobacco smoke, dietary aflatoxins) have been associated with particular types of mutations. Inherited mutations are, in their majority, transitions at CpG dinucleotides (54%) or small deletions/insertions (23%) that may occur spontaneously rather than as consequences of carcinogen exposures.

The detection of DNA mutations could also be performed using different DNA chips or oligonucleotide probe microarrays technologies (Affimetrix).

The presence of a loss-of-function mutation in one of the genes known to regulate the AID mutator activity in a B cell (e.g., p53, ATM, Nbs1, UNG, SMUG1, MSH2 and MSH6) will be indicative that the administration of an Hsp90 inhibitor (e.g., 17-AAG) is appropriate.

In parallel, the gene expression analysis will be performed. A B cell population sample will be obtained from a patient and the level of mRNA expression for genes known to regulate the AID mutator activity (e.g., p53, ATM, Nbs1, UNG, SMUG1, MSH2 and/or MSH6) will be evaluated. The measurement will be performed, according to standard PCR conditions, by quantitative real-time PCR carried out with the SYBR™ Green ER mix from Invitrogen (Carlsbad, Calif.) using primers specific for each mRNAs. During the PCR amplification, the SYBR™ Green ER dye in the mix binds to accumulating double-stranded DNA and generates a fluorescence signal proportional to the DNA concentration that can be visualized and measured using a ABI7900HT (Applied Biosystems, Foster City, Calif.) real-time PCR system.

The levels of RNA measured in the patient sample will be compared to the levels from control samples obtained from patients that do not have this disease and/or are not likely to develop the disease. As a control cell population, normal naive B cells (e.g., CD19+CD27+IgD+) will be sorted from peripheral blood of healthy donors by flow cytometry using a FACS Vantage™ SE cell sorter (BD Biosciences).

A lower level of expression of one of the genes known to regulate the AID mutator activity in a B cell (e.g., p53, ATM, Nbs1, UNG, SMUG1, MSH2 and/or MSH6) in the patient sample as compared to the reference expression of that gene (e.g., a statistically significant reduction of 2-fold or more)) will be indicative that the administration of an Hsp90 inhibitor (e.g., 17-AAG) is appropriate.

### Example 11

### Treatment of Patients Having Clinical Manifestations of Allergy with an Hsp90 Inhibitor

A subject having clinical manifestations of allergic rhinitis will be topically treated with nasal spray containing an Hps90 inhibitor.

### REFERENCES

1 Neuberger, M. S. Antibody diversification by somatic mutation: from Burnet onwards. Immunol Cell Biol 86, 124-132 (2008).
2 Di Noia, J. M. & Neuberger, M. S. Molecular mechanisms of antibody somatic hypermutation. Annu Rev Biochem 76, 1-22 (2007).
3 Neuberger, M. S. et al. Memory in the B-cell compartment: antibody affinity maturation. Philos Trans R Soc Lond B Biol Sci 355, 357-360, doi:10.1098/rstb.2000.0573 (2000).
4 Muramatsu, M. et al. Class switch recombination and hypermutation require activation-induced cytidine deaminase (AID), a potential RNA editing enzyme. Cell 102, 553-563 (2000).
5 Peled, J. U. et al. The Biochemistry of Somatic Hypermutation. Annu Rev Immunol (2007).
6 Stavnezer, J., Guikema, J. E. J. & Schrader, C. E. Mechanism and Regulation of Class Switch Recombination. Annu Rev Immunol 26, 261-292, doi:10.1146/annurev.immuno1.26.021607.090248 (2008).
7 Muramatsu, M. et al. Specific expression of activation-induced cytidine deaminase (AID), a novel member of the RNA-editing deaminase family in germinal center B cells. J Biol Chem 274, 18470-18476 (1999).
8 Macduff, D., Demorest, Z. & Harris, R. AID can restrict L1 retrotransposition suggesting a dual role in innate and adaptive immunity. Nucleic Acids Res, doi:10.1093/nar/gkp030 (2009).
9 Pauklin, S., Sernandez, I., Bachmann, G., Ramiro, A. R. & Petersen-Mahrt, S. K. Estrogen directly activates AID transcription and function. J Exp Med 206, 99-111, doi:10.1084/jem.20080521 (2009).
10 Sernandez, I. V., De Yebenes, V. G., Dorsett, Y. & Ramiro, A. R. Haploinsufficiency of activation-induced deaminase for antibody diversification and chromosome translocations both in vitro and in vivo. PLoS ONE 3, e3927, doi:10.1371/journal.pone.0003927 (2008).
11 Takizawa, M. et al. AID expression levels determine the extent of cMyc oncogenic translocations and the incidence of B cell tumor development. J Exp Med 205, 1949-1957, doi:10.1084/jem.20081007 (2008).
12 de Yebenes, V. et al. miR-181b negatively regulates activation-induced cytidine deaminase in B cells. J Exp Med, doi:10.1084/jem.20080579 (2008).
13 Dorsett, Y. et al. MicroRNA-155 suppresses activation-induced cytidine deaminase-mediated Myc-Igh translocation. Immunity 28, 630-638, doi:10.1016/j.immuni.2008.04.002 (2008).
14 Teng, G. et al. MicroRNA-155 is a negative regulator of activation-induced cytidine deaminase. Immunity 28, 621-629, doi:10.1016/j.immuni.2008.03.015 (2008).
15 Robbiani, D. F. et al. AID Produces DNA Double-Strand Breaks in Non-Ig Genes and Mature B Cell Lymphomas with Reciprocal Chromosome Translocations. Mol Cell 36, 631-641, doi:10.1016/j.molce1.2009.11.007 (2009).
16 Ramiro, A. R. et al. AID is required for c-myc/IgH chromosome translocations in vivo. Cell 118, 431-438 (2004).
17 Gostissa, M. et al. Long-range oncogenic activation of Igh-c-myc translocations by the Igh 3′ regulatory region. Nature 462, 803-807 (2009).
18 Okazaki, I. M. et al. Constitutive expression of AID leads to tumorigenesis. J Exp Med 197, 1173-1181 (2003).
19 Greeve, J. et al. Expression of activation-induced cytidine deaminase in human B-cell non-Hodgkin lymphomas. Blood 101, 3574-3580, doi:10.1182/blood-2002-08-2424 (2003).
20 Pasqualucci, L. et al. Expression of the AID protein in normal and neoplastic B cells. Blood 104, 3318-3325 (2004).
21 Albesiano, E. et al. Activation-induced cytidine deaminase in chronic lymphocytic leukemia B cells: expression as multiple forms in a dynamic, variably sized fraction of the clone. Blood 102, 3333-3339, doi:10.1182/blood-2003-05-1585 (2003).
22 Feldhahn, N. et al. Activation-induced cytidine deaminase acts as a mutator in BCR-ABL1-transformed acute lymphoblastic leukemia cells. J Exp Med 204, 1157-1166, doi:10.1084/jem.20062662 (2007).
23 Klemm, L. et al. The B cell mutator AID promotes B lymphoid blast crisis and drug resistance in chronic myeloid leukemia. Cancer Cell 16, 232-245, doi:10.1016/j.ccr.2009.07.030 (2009).
24 Endo, Y. et al. Activation-Induced Cytidine Deaminase Links Between Inflammation and the Development of Colitis-Associated Colorectal Cancers. Gastroenterology, doi:10.1053/j.gastro.2008.06.091 (2008).
25 Kou, T. et al. Expression of activation-induced cytidine deaminase in human hepatocytes during hepatocarcinogenesis. Int J Cancer 120, 469-476, doi:10.1002/ijc.22292 (2007).
26 Matsumoto, Y. et al. Helicobacter pylori infection triggers aberrant expression of activation-induced cytidine deaminase in gastric epithelium. Nat Med 13, 470-476, doi:10.1038/nm1566 (2007).
27 Ramiro, A. R. et al. Role of genomic instability and p53 in AID-induced c-myc-Igh translocations. Nature 440, 105-109 (2006).
28 Patenaude, A. M. et al. Active nuclear import and cytoplasmic retention of activation-induced deaminase. Nat Struct Mol Biol 16, 517-527, doi:nsmb.1598 [pii] 10.1038/nsmb.1598 (2009).
29 Conticello, S. G. et al. Interaction between antibody-diversification enzyme AID and spliceosome-associated factor CTNNBL1. Mol Cell 31, 474-484 (2008).
30 Prochnow, C., Bransteitter, R., Klein, M., Goodman, M. F. & Chen, X. The APOBEC-2 crystal structure and functional implications for the deaminase AID. Nature 445, 447-451 (2006).
31 Kim, C. J. et al. Activation-induced cytidine deaminase expression in gastric cancer. Tumour Biol 28, 333-339 (2007).
32 Komori, J. et al. Activation-induced cytidine deaminase links bile duct inflammation to human cholangiocarcinoma. Hepatology 47, 888-896 (2008).
33 Leuenberger, M. et al. AID protein expression in chronic lymphocytic leukemia/small lymphocytic lymphoma is associated with poor prognosis and complex genetic alterations. Mod Pathol 23, 177-186 (2010).
34 Chiba, T. & Marusawa, H. A novel mechanism for inflammation-associated carcinogenesis; an important role of activation-induced cytidine deaminase (AID) in mutation induction. J Mol Med 87, 1023-1027 (2009).
35 Jankovic, M. et al. Role of the translocation partner in protection against AID-dependent chromosomal translocations. Proc Natl Acad Sci USA (2009).
36 Liu, M. et al. Two levels of protection for the B cell genome during somatic hypermutation. Nature 451, 841-845 (2008).
37 Rada, C., Di Noia, J. M. & Neuberger, M. S. Mismatch recognition and uracil excision provide complementary paths to both Ig switching and the NT-focused phase of somatic mutation. Mol Cell 16, 163-171 (2004).
38 Saribasak, H. et al. Uracil DNA glycosylase disruption blocks Ig gene conversion and induces transition mutations. J Immunol 176, 365-371 (2006).
39 Shen, H. M., Tanaka, A., Bozek, G., Nicolae, D. & Storb, U. Somatic hypermutation and class switch recombination in Msh6(−/−)Ung(−/−) double-knockout mice. J Immunol 177, 5386-5392 (2006).
40 Wu, X. & Stavnezer, J. DNA polymerase beta is able to repair breaks in switch regions and plays an inhibitory role during immunoglobulin class switch recombination. J Exp Med 204, 1677-1689 (2007).
41 Nilsen, H. et al. Gene-targeted mice lacking the Ung uracil-DNA glycosylase develop B-cell lymphomas. Oncogene 22, 5381-5386 (2003).
42 Coll-Mulet, L. & Gil, J. Genetic alterations in chronic lymphocytic leukaemia. Clin Transl Oncol 11, 194-198, doi:1202 [pii] (2009).
43 Zenz, T. et al. Treatment resistance in chronic lymphocytic leukemia: the role of the p53 pathway. Leuk Lymphoma 50, 510-513, doi:910217998 [pii] 10.1080/10428190902763533 (2009).
44 Smit, L. A. et al. Expression of activation-induced cytidine deaminase is confined to B-cell non-Hodgkin's lymphomas of germinal-center phenotype. Cancer Research 63, 3894-3898 (2003).
45 Cohen-Solal, J. F. G., Jeganathan, V., Grimaldi, C. M., Peeva, E. & Diamond, B. Sex hormones and SLE: influencing the fate of autoreactive B cells. Curr Top Microbiol Immunol 305, 67-88 (2006).
46 Cohen-Solal, J. F. G. et al. Hormonal regulation of B-cell function and systemic lupus erythematosus. Lupus 17, 528-532 (2008).
47 Hsu, H.-C. et al. Overexpression of activation-induced cytidine deaminase in B cells is associated with production of highly pathogenic autoantibodies. J Immunol 178, 5357-5365 (2007).
48 Jiang, C. et al. Abrogation of lupus nephritis in activation-induced deaminase-deficient MRL/lpr mice. J Immunol 178, 7422-7431 (2007).
49 Goodnow, C. C. Multistep pathogenesis of autoimmune disease. Cell 130, 25-35 (2007).
50 Coker, H. A., Durham, S. R. & Gould, H. J. Local somatic hypermutation and class switch recombination in the nasal mucosa of allergic rhinitis patients. J Immunol 171, 5602-5610 (2003).
51 Takhar, P. et al. Allergen drives class switching to IgE in the nasal mucosa in allergic rhinitis. J Immunol 174, 5024-5032 (2005).
52 Ito, M. et al. Enhanced expression of lymphomagenesis-related genes in peripheral blood B cells of chronic hepatitis C patients. Clin Immunol (2010).
53 Machida, K. et al. Hepatitis C virus induces a mutator phenotype: enhanced mutations of immunoglobulin and protooncogenes. Proc Natl Acad Sci USA 101, 4262-4267 (2004).
54 Epeldegui, M. et al. Elevated expression of activation induced cytidine deaminase in peripheral blood mononuclear cells precedes AIDS-NHL diagnosis. AIDS 21, 2265-2270, doi:10.1097/QAD.0b013e3282ef9f59 (2007).
55 Epeldegui, M., Hung, Y. P., McQuay, A., Ambinder, R. F. & Martinez-Maza, O. Infection of human B cells with Epstein-Barr virus results in the expression of somatic hypermutation-inducing molecules and in the accrual of oncogene mutations. Mol Immunol 44, 934-942 (2007).
56 Takai, A. et al. A novel mouse model of hepatocarcinogenesis triggered by AID causing deleterious p53 mutations. Oncogene 28, 469-478 (2009).
57 Durandy, A., Peron, S., Taubenheim, N. & Fischer, A. Activation-induced cytidine deaminase: structure-function relationship as based on the study of mutants. Hum Mutat 27, 1185-1191 (2006).
58 Ito, S. et al. Activation-induced cytidine deaminase shuttles between nucleus and cytoplasm like apolipoprotein B mRNA editing catalytic polypeptide 1. Proc Natl Acad Sci USA 101, 1975-1980 (2004).
59 McBride, K. M., Barreto, V., Ramiro, A. R., Stavropoulos, P. & Nussenzweig, M. C. Somatic hypermutation is limited by CRM1-dependent nuclear export of activation-induced deaminase. J Exp Med 199, 1235-1244, doi:10.1084/jem.20040373 (2004).
60 Aoufouchi, S. et al. Proteasomal degradation restricts the nuclear lifespan of AID. J Exp Med 205, 1357-1368, doi:10.1084/jem.20070950 (2008).
61 Csermely, P., Schnaider, T., Soti, C., Prohászka, Z. & Nardai, G. The 90-kDa molecular chaperone family: structure, function, and clinical applications. A comprehensive review. Pharmacol Ther 79, 129-168 (1998).
62 Hutchison, K. A., Dittmar, K. D. & Pratt, W. B. All of the factors required for assembly of the glucocorticoid receptor into a functional heterocomplex with heat shock protein 90 are preassociated in a self-sufficient protein folding structure, a “foldosome”. J Biol Chem 269, 27894-27899 (1994).
63 Pearl, L. H. & Prodromou, C. Structure and mechanism of the Hsp90 molecular chaperone machinery. Annu Rev Biochem 75, 271-294, doi:10.1146/annurev.biochem. 75.103004.142738 (2006).
64 Picard, D. Chaperoning steroid hormone action. Trends Endocrinol Metab 17, 229-235, doi:10.1016/j.tem.2006.06.003 (2006).
65 Wandinger, S. K., Richter, K. & Buchner, J. The Hsp90 chaperone machinery. J Biol Chem 283, 18473-18477, doi:10.1074/jbc.R800007200 (2008).
66 Whitesell, L. & Lindquist, S. L. HSP90 and the chaperoning of cancer. Nat Rev Cancer 5, 761-772, doi:10.1038/nrc1716 (2005).
67 Shelton, S, N. et al. KU135, a novel novobiocin-derived C-terminal inhibitor of the 90-kDa heat shock protein, exerts potent antiproliferative effects in human leukemic cells. Mol Pharmacol 76, 1314-1322, doi:mol.109.058545 [pii] 10.1124/mol.109.058545 (2009).
68 Matthews, S. B. et al. Characterization of a novel novobiocin analogue as a putative C-terminal inhibitor of heat shock protein 90 in prostate cancer cells. Prostate 70, 27-36, doi:10.1002/pros.21035 (2010).
69 Rossi, D. et al. Aberrant somatic hypermutation in transformation of follicular lymphoma and chronic lymphocytic leukemia to diffuse large B-cell lymphoma. Haematologica 91, 1405-1409 (2006).
70 Oppezzo, P. et al. Chronic lymphocytic leukemia B cells expressing AID display dissociation between class switch recombination and somatic hypermutation. Blood 101, 4029-4032 (2003).
71 Endo, Y. et al. Expression of activation-induced cytidine deaminase in human hepatocytes via NF-kappaB signaling. Oncogene 26, 5587-5595 (2007).
72 Babbage, G., Ottensmeier, C. H., Blaydes, J., Stevenson, F. K. & Sahota, S. S. Immunoglobulin heavy chain locus events and expression of activation-induced cytidine deaminase in epithelial breast cancer cell lines. Cancer Res 66, 3996-4000 (2006).
73 Porter, J. R., Ge, J., Lee, J., Normant, E. & West, K. Ansamycin inhibitors of Hsp90: nature's prototype for anti-chaperone therapy. Curr Top Med Chem 9, 1386-1418, doi:CTMC-Abs-027-9-15 [pii] (2009).
74 Kim, Y. S. et al. Update on Hsp90 inhibitors in clinical trial. Curr Top Med Chem 9, 1479-1492, doi:CTMC-Abs-031-9-15 [pii] (2009).
75 Tauchi, T. & Ohyashiki, K. Imatinib mesylate in combination with other chemotherapeutic agents for chronic myelogenous leukemia. Int J Hematol 79, 434-440 (2004).
76 Di Noia, J. M. et al. Dependence of antibody gene diversification on uracil excision. J Exp Med 204, 3209-3219 (2007).
77 Dedeoglu, F., Horwitz, B., Chaudhuri, J., Alt, F. W. & Geha, R. S. Induction of activation-induced cytidine deaminase gene expression by IL-4 and CD40 ligation is dependent on STAT6 and NFkappaB. Int Immunol 16, 395-404 (2004).
78 Nakamura, M. et al. High frequency class switching of an IgM+B lymphoma clone CH12F3 to IgA+ cells. Int Immunol 8, 193-201 (1996).
79 Arakawa, H., Hauschild, J. & Buerstedde, J.-M. Requirement of the activation-induced deaminase (AID) gene for immunoglobulin gene conversion. Science 295, 1301-1306, doi:10.1126/science.1067308 (2002).
80 Di Noia, J. M. & Neuberger, M. S. Immunoglobulin gene conversion in chicken DT40 cells largely proceeds through an abasic site intermediate generated by excision of the uracil produced by AID-mediated deoxycytidine deamination. Eur J Immunol 34, 504-508 (2004).
81 Arakawa, H., Saribasak, H. & Buerstedde, J.-M. Activation-induced cytidine deaminase initiates immunoglobulin gene conversion and hypermutation by a common intermediate. PLoS Biol 2, E179, doi:10.1371/journal.pbio.0020179 (2004).
82 Sreedhar, A. S., Kalmar, E., Csermely, P. & Shen, Y. F. Hsp90 isoforms: functions, expression and clinical importance. FEBS Lett 562, 11-15 (2004).
83 Hansen, L. K., Houchins, J. P. & O'Leary, J. J. Differential regulation of HSC70, HSP70, HSP90 alpha, and HSP90 beta mRNA expression by mitogen activation and heat shock in human lymphocytes. Exp Cell Res 192, 587-596 (1991).
84 Metz, K., Ezernieks, J., Sebald, W. & Duschl, A. Interleukin-4 upregulates the heat shock protein Hsp90alpha and enhances transcription of a reporter gene coupled to a single heat shock element. FEBS Lett 385, 25-28 (1996).
85 Conticello, S. G., Thomas, C. J., Petersen-Mahrt, S. K. & Neuberger, M. S. Evolution of the AID/APOBEC family of polynucleotide (deoxy)cytidine deaminases. Mol Biol Evol 22, 367-377 (2005).
86 Dickey, C. A. et al. The high-affinity HSP90-CHIP complex recognizes and selectively degrades phosphorylated tau client proteins. J Clin Invest 117, 648-658, doi:10.1172/JC129715 (2007).
87 Panaretou, B. et al. ATP binding and hydrolysis are essential to the function of the Hsp90 molecular chaperone in vivo. EMBO J. 17, 4829-4836, doi:10.1093/emboj/17.16.4829 (1998).
88 Young, J. C. & Hartl, F. U. Polypeptide release by Hsp90 involves ATP hydrolysis and is enhanced by the co-chaperone p23. EMBO J. 19, 5930-5940, doi:10.1093/emboj/19.21.5930 (2000).
89 DeFranco, D. B. Regulation of steroid receptor subcellular trafficking. Cell Biochem Biophys 30, 1-24, doi:10.1007/BF02737882 (1999).
90 Galigniana, M. D., Harrell, J. M., O'Hagen, H. M., Ljungman, M. & Pratt, W. B. Hsp90-binding immunophilins link p53 to dynein during p53 transport to the nucleus. J Biol Chem 279, 22483-22489, doi:10.1074/jbc.M402223200 (2004).
91 Barreto, V. M., Reina-San-Martin, B., Ramiro, A. R., McBride, K. M. & Nussenzweig, M. C. C-terminal deletion of AID uncouples class switch recombination from somatic hypermutation and gene conversion. Mol Cell 12, 501-508 (2003).
92 Young, J. C., Agashe, V. R., Siegers, K. & Hartl, F. U. Pathways of chaperone-mediated protein folding in the cytosol. Nat Rev Mol Cell Biol 5, 781-791, doi:10.1038/nrm1492 (2004).
93 McDonough, H. & Patterson, C. CHIP: a link between the chaperone and proteasome systems. Cell Stress Chaperones 8, 303-308 (2004).
94 Li, L. et al. CHIP mediates degradation of Smad proteins and potentially regulates Smad-induced transcription. Molecular and Cellular Biology 24, 856-864 (2004).
95 Borkovich, K. A., Farrelly, F. W., Finkelstein, D. B., Taulien, J. & Lindquist, S. hsp82 is an essential protein that is required in higher concentrations for growth of cells at higher temperatures. Molecular and Cellular Biology 9, 3919-3930 (1989).
96 Cutforth, T. & Rubin, G. M. Mutations in Hsp83 and cdc37 impair signaling by the sevenless receptor tyrosine kinase in Drosophila. Cell 77, 1027-1036 (1994).
97 Schulte, T. W. & Neckers, L. M. The benzoquinone ansamycin 17-allylamino-17-demethoxygeldanamycin binds to HSP90 and shares important biologic activities with geldanamycin. Cancer Chemother Pharmacol 42, 273-279 (1998).
97 Hodgkin, P. D., Lee, J. H. & Lyons, A. B. B cell differentiation and isotype switching is related to division cycle number. J Exp Med 184, 277-281 (1996).
99. Rush, J. S., Liu, M., Odegard, V. H., Unniraman, S. & Schatz, D. G. Expression of activation-induced cytidine deaminase is regulated by cell division, providing a mechanistic basis for division-linked class switch recombination. Proc Natl Acad Sci USA 102, 13242-13247, doi:10.1073/pnas.0502779102 (2005).
100. Petitjean, A. et al. Impact of mutant p53 functional properties on TP53 mutation patterns and tumor phenotype: lessons from recent developments in the IARC TP53 database. Hum Mutat 28, 622-629, doi:10.1002/humu.20495 (2007).
101. Brandford, S., and Hughes, T. Detection of BCR-ABL mutations and resistance to imatinib mesylate. Methods Mol. Med. 9, 3919-3930 (2006).

1. A method for stratifying a subject, said method comprising:
a. measuring the AID expression and/or activity in a first sample of the subject, and
b. comparing said expression and/or activity to a reference AID expression and/or activity, wherein an AID expression and/or activity in the first sample of the subject that is higher than the reference AID expression and/or activity is indicative that the subject would benefit from a treatment with at least one Heat Shock Protein 90 (Hsp90) inhibitor.

2. The method of claim 1, wherein when the AID expression in the first sample of the subject is substantially similar to the reference AID expression, the method further comprises the step of:
c. detecting in the first or a second sample of the subject the presence of a loss-of-function mutation in at least one gene known to regulate AID mutator activity by controlling or repairing DNA damage,
wherein the presence of a mutation in the at least one gene in the first or second sample of the subject is indicative that the subject would benefit from a treatment with at least one Heat Shock Protein 90 (Hsp90) inhibitor.

3. A method for the prevention and/or treatment of an AID-associated disease in a subject in need thereof, said method comprising:
a. measuring the level of AID expression and/or activity in a first sample from the subject,
b. comparing said expression and/or activity to a reference AID expression and/or activity,
wherein, if the AID expression and/or activity is higher in the first sample from the subject than the reference AID expression and/or activity, an effective amount of an Heat Shock Protein 90 (Hsp90) inhibitor is administered to the patient.

4. The method of claim 3, wherein when the AID expression in the first sample of the subject is substantially similar to the reference AID expression, the method further comprises the step of:
c. detecting in the first or a second sample of the subject the presence of a loss-of-function mutation in at least one gene known to regulate AID mutator activity by controlling or repairing DNA damage,
wherein the presence of a mutation in the at least one gene in the first or second sample of the subject is indicative that the subject would benefit from a treatment with at least one Heat Shock Protein 90 (Hsp90) inhibitor.

5. The method of claim 3, wherein the AID-associated disease is cancer and the sample from the subject is pre neoplastic or neoplastic tissue.

6. The method of claim 5, wherein the cancer is an immune system cancer or a solid tumor.

7. The method of claim 6, where the immune system cancer is chronic myeloid leukemia (CML) or BCR-ABL1-positive acute lymphoid leukemia (ALL).

8. The method of claim 6, where the solid tumor is Helicobacter pylori-associated gastric tumor, liver tumor or colorectal cancer tumor.

9. The method of claim 1, wherein the AID-associated disease is an autoimmune disease, and the sample from the subject is a B lymphocyte population of the subject.

10. A method for preventing drug resistance in a subject having an AID-expressing neoplastic disease, said method comprising:
a. measuring the level of AID expression and/or activity in a tissue sample from the subject, and
b. administering an effective amount of an Hsp90 inhibitor in combination with the drug, to the subject having an AID-positive tissue,
whereby the drug resistance is prevented.

11. The method of claim 10, wherein the neoplastic disease is chronic myeloid leukemia.

12. The method of claim 11, wherein the drug is imatinib.

13. The method of claim 3, wherein the Hsp90 inhibitor is a geldanamycin analog.

14. The method of claim 13, wherein the geldanamycin analog is 17-(Allylamino)-17-demethoxygeldanamycin (17-MG), 17-(Dimethylaminoethylamino)-17-demethoxygeldanamycin (17-DMAG), nab-17-AAGs, NXD30001 or CNF1010.

15. The method of claim 3, wherein the administration is a monotherapy.

16. The method of claim 3, further comprising administration of at least one other therapy to the subject.

17. The method of claim 16, wherein the at least one other therapy comprises at least one further AID inhibitor.

18. The method of claim 17, wherein the at least one AID inhibitor is not an Hsp90 inhibitor.

19. The method of claim 3, further comprising administration of at least one further anticancer treatment.

20. The method of claim 3, wherein the subject is undergoing a therapy that comprises the administration of least one compound that increases AID expression and/or activity in a normal tissue.

21. The method of claim 20, where the compound is estrogen.

22. A method for adjusting a dose of a Hsp90 inhibitor in a treatment, said method comprising:
a. measuring the level of AID expression and/or activity in a sample of a subject treated with an Hsp90 inhibitor,
b. comparing said expression and/or activity to a reference AID expression and/or activity from the subject at an earlier time, and
c. administering to the subject having a substantially similar or higher AID expression and/or activity than the reference AID expression and/or activity an increased dose of the Hsp90 inhibitor.

23. A kit for preventing and/or treating an AID-associated disease or for stratifying a subject having an AID-associated disease comprising an AID ligand and a Heat Shock Protein 90 (Hsp90) inhibitor.

24. The kit of claim 23, wherein the AID-associated disease is a neoplastic disease and further comprising a further antitumoral agent.
